(19) United States
(12) Patent Application Publication
(10) Pub. No.: US 2019/0362846 A1
(43) Pub. Date: Nov. 28, 2019

(54) SYSTEM AND METHOD FOR AN AUTOMATED CLINICAL DECISION SUPPORT SYSTEM

(71) Applicant: Siemens Healthcare GmbH, Erlangen (DE)

(72) Inventor: Asmir VODENCAREVIC, Fuerth (DE)

(73) Assignee: Siemens Healthcare GmbH, Erlangen (DE)

(21) Appl. No.: 16/413,769

(22) Filed: May 16, 2019

(30) Foreign Application Priority Data
May 24, 2018 (EP) 18174108.3
Jun. 29, 2018 (EP) 18180907.0
Aug. 3, 2018 (EP) 18187263.1

(51) Int. Cl. G16H 50/20; G16H 50/30; G16H 10/60

(52) U.S. Cl. CPC G16H 50/20; G16H 50/70; G16H 10/60; G16H 50/30

(57) ABSTRACT

A method is for creating predictive models for an automated clinical decision support system for automated supervised and semi-supervised classification and treatment optimization of clinical events, e.g. of disease activity in autoimmune diseases, using EMR data and predictive models in a nested cross validation, as well as a respective prediction-unit for creating prediction-data for an automated clinical decision support system. Another method is for automated clinical decision support for automated supervised and semi-supervised classification and treatment optimization of clinical events using EMR data, as well as a respective decision support system.

### PRIORITY STATEMENT

The present application hereby claims priority under 35 U.S.C. § 119 to European patent application numbers EP18187263.1 filed Aug. 3, 2018; EP18180907.0 filed Jun. 29, 2018; and EP 18174108.3 filed May 24, 2018, the entire contents of each of which are hereby incorporated herein by reference.

### FIELD

Embodiments of the invention generally relate to a method for creating predictive models for an automated clinical decision support system for automated supervised and semi-supervised classification and treatment optimization of clinical events, e.g. of disease activity in autoimmune diseases, using EMR data, as well as a prediction-unit for creating prediction-data for an automated clinical decision support system. Embodiments of the invention further generally relate to a method for automated clinical decision support for automated supervised and semi-supervised classification and treatment optimization of clinical events using EMR data, as well as a respective clinical decision support system.

### BACKGROUND

Electronic Medical Records (EMRs) of patients represent systematized collections of patients' health data. They are populated in a clinical routine in a longitudinal manner through interactions of the patients with healthcare providers. Electronic medical records can contain different types of data, comprising demographic data, laboratory measurements over time, administered medications, examination data, clinical notes and images (e.g. from X-ray imaging, from ultrasound devices, from computed tomography or magnetic resonance imaging). On the patient population level (e.g. patient population diagnosed with a specific autoimmune disease such as Rheumatoid Arthritis (an acronym is “RA”)), electronic medical records are large data repositories that can be utilized by data-driven approaches such as machine learning classification algorithms in various practical use cases. One exemplary use case is using the EMR data of patients diagnosed with RA to estimate the probability of flares within a certain time horizon (e.g. 3 months or 6 months). Such data-driven approaches require the data to be in a tabular format with columns representing different variables (e.g. patient age, gender, lab measurements) and rows representing patient follow-ups during which various data is collected. Moreover, the data has to be complete (i.e. missing values have to be treated in some way) and the label has to be available (i.e. whether a flare has occurred or not within an observed time horizon).

In general there are different problems for applying data-driven approaches to electronic medical records:

(A) The EMR data is collected at irregular time intervals which are different for different patients (some patients have many more follow-ups than others). Not all in the EMR available fields/variables are collected/measured/entered at each follow-up resulting in a scarcely populated EMR (i.e. many missing values). In the RA example, this is especially the case with many relevant variables such as the CRP laboratory measurement. Such scenario makes it impossible to apply statistical or machine learning approaches to predictive modeling as they rely on data in a tabular format and most of them cannot work directly with the missing values. Moreover, even if a value is available for the current follow-up, it is temporally related to previously observed values that should be analyzed in the common context.

(B) EMRs often contain hidden structures in the data which have predictive power with respect to some classification task (e.g. predicting RA flare occurrence within a certain time horizon). However, these structures are not obvious or directly accessible in the original multidimensional data space and therefore cannot be used explicitly in the modeling task.

(C) Electronic medical records can include many variables making it difficult to keep an overview of the disease dynamics, i.e. changes in a specific disease activity over time.

(D) When using electronic medical records of a patient population for building a classifier to predict the future disease activity (e.g. building classifiers for predicting the flare probability in RA patients), the follow-ups need to include a label about the disease activity (in the RA example the label shows whether the flares are observed within a certain time horizon after that follow-up or not) which is often not available or expensive/time-consuming to obtain. In some observed real-world cases, the label is missing in about ⅔ of the follow-ups.

(E) When building classifiers for autoimmune diseases from the EMR data, no free lunch theorem holds, i.e. there is no way to know a priori which type of an algorithm and with what hyperparameter values would work the best on the given problem. This causes long development times of such classifiers as various options need to be evaluated and compared. By using certain model selection procedures it would be possible to partially automate this model selection procedure; however it is not obvious how such procedure could work when the available EMR datasets contain both labeled and unlabeled data.

(F) Clinicians treating autoimmune diseases don't have an insight in trade-offs between various predicted risks of adverse events (such as e.g. RA flares or even death within different time horizons) and expenses associated with their treatment decisions including follow-up frequency. With such trade-offs, an implementation of the clinical decision support system giving an optimal treatment recommendations would be possible. Moreover, different clinicians prefer different treatment strategies, i.e. some are more conservative and some more liberal in treating the disease which makes it hard to establish a common generally acceptable standard for treatment recommendations.

Various approaches are known for the person skilled in the art in order to deal with the described problems:

(A) It is known to treat missing values by imputation methods such as mean, median, regression imputation and multiple imputations, where missing values are estimated using known values. Another approach is to encode the missing values by binarization. If the variable is categorical with N levels (e.g. gender has N=2 levels: male and female), binarization approach creates two binary variables where the first one has the value 1 for those patients who are male and zero for those who are female. Likewise, the second variable has the value 1 for the female patients and value 0 otherwise. In case the value is missing for a patient, his/her gender is encoded with values 0 in both binary variables. If the variable is numerical, one or more cutting points are defined based on some statistics, e.g. based on the median value (or based on 33% and 67% percentiles for two thresholds or similar). For a threshold based on these percentiles, three binary variables are created, the first one containing values ‘1’ at positions where corresponding numerical value is lower than or equal to the 33% percentile (measurement low value), the second one containing ‘1’ where corresponding numerical value is higher than 33% percentile and lower or equal to the 66% percentile (measurement normal value), and the third one containing ‘1’ where the corresponding numerical value is higher than the 66% percentile. If the numerical value is missing, all three binary variables receive value 0. FIG. 1 illustrates this approach used to encode categorical medication variables as well as the numerical laboratory values.

Temporal dependencies of clinical events or measurements can be modeled using weighting functions within sliding windows. Namely, a time frame is defined based on the relevancy of the past data for the current disease activity. This time frame represents the length of the sliding window that defines the weighting function. Such function has to be monotonically decreasing (e.g. linearly or exponentially, see FIG. 2), having its maximum value of 1 at the most recent follow-up recorded in the patient's EMR and its minimum value of 0 at the last time point within the relevancy time frame. For each current visit, the currently measured value is replaced by a sum of relevant past values multiplied by their corresponding weights. However, from the prior art it is unknown how to address the missing value problem, and in general the applied method produces unrealistic and for clinicians uninterpretable feature values (e.g. typical CRP blood value could be replaced by a weighted sum of relevant past values which can be easily outside of the possible CRP values).

(B) It is known to apply clustering to identify subgroups/hidden structures of patients from their EMR data. However clustering EMR follow-ups of patients with certain disease in order to compute cluster features and them in predictive modeling of a disease activity has not been applied to the EMR data in the prior art.

(C) It is known to apply dimensionality reduction using methods such as Principal Component Analysis (PCA) for the purpose of visualization to the EMR. PCA is a technique for dimensionality reduction that can reduce the EMR data to two or three dimensions (called principal components), which can be easily visualized. However, in the known prior art this is done in a static way, i.e. each visualized data point represents one patient in a new PCA space. This enables visualization of patients; however this visualization is static, not revealing changes in patients' health over time. It is known to use the PCA approach in a dynamic way in other technical areas, namely in predictive maintenance of technical systems such as gas turbine engines and jet engines, which is illustrated in FIG. 3.

(D) It is known to treat follow-ups with missing labels by excluding them from the analysis. However, this can cause big loss of the valuable data. Furthermore it is known to treat missing labels in patient EMRs by denoising autoencoders. In this setting, both available labeled and unlabeled data are used to train a denoising autoencoder (a type of deep learning neural network). The unlabeled data is then discarded and the hidden layer of the denoising autoencoder is used as the input to classical supervised learning methods such as a random forest. Therefore, in this approach the unlabeled instances are NOT labeled but rather used in an unsupervised manner to improve tuning neurons in the hidden layer of the denoising autoencoder. Moreover, since hidden layer performs dimensionality reduction it provides abstract features to the classifier, which are not understandable to or interpretable by clinicians.

(E) It is known to perform selection and hyperparameter optimization in predictive modeling using a procedure called k-fold cross-validation. In this procedure, the available dataset is divided in k mutually exclusive partitions, mostly by random sampling without replacement. In the first fold, k−1 partitions are used for training a model which is then evaluated on the remaining partition. This is repeated k times, each time having different k-th set as a test set. In each fold, some performance metric is computed, such as the Area Under the receiver operating Characteristics (AUC) or classification error. Afterwards, the mean value and the standard deviation of the performance metrics are computed over k folds. For each model and for each set of hyperparameters such a k-fold cross validation procedure is performed, averaged results of the k-folds are compared for different models (and/or hyperparameter sets) and finally the one that maximized the model performance is selected as the best model. The illustration of the k-fold cross-validation is given in FIG. 4.

However, the performance computed in this way can be significantly biased, as the results on the k-test sets are actually used to make decisions about models and/or hyperparameter values (i.e. it is known that the problem of overfitting can occur). In order to mitigate this problem, a nested k-fold cross-validation procedure can be used, which provides an almost unbiased estimate of the true performance on unseen data (see e.g. Semi-Supervised Learning; edited by Olivier Chapelle, Bernhard Schölkopf, and Alexander Zien). In short, the nested cross-validation consists of two k-fold loops (note that loops can have different values of the ‘k’ parameter): the inner one which tunes the hyperparameters and the outer one which estimates the performance. The application of the nested cross-validation for model selection and hyperparameter optimization is straightforward: a number of supervised learning algorithms and their corresponding hyperparameter values are defined in a grid and evaluated in a nested cross-validation procedure w.r.t. some performance metrics of interest. At the end, the best model and its parameter set is selected. The procedure is illustrated in FIG. 5.

(F) It is known to compare a therapy selected by a clinician or a nurse to the therapy recommended by the proposed clinical decision support system based on the machine learning model (i.e. the output of the machine learning model is a therapy recommendation). Applied machine learning modeling formalism is the recurrent neural network. In this approach, the most likely therapy is directly modeled as an output of the machine learning task and moreover, the considered optimization problem relates to the therapy type only without considering additional factors such as the therapy expense and its influence on the patient follow-up frequency. Furthermore, multi-objective optimization is known which takes into account the following criteria: therapy timing, type and expense with constraints imposed on the type and maximum allowed expense. It is a numerical optimization problem which doesn't rank the importance of these single optimization criteria for clinicians. Moreover, it doesn't provide insights into trade-offs between these criteria.

### SUMMARY

Concerning EMR-data, the inventors have discovered that there is the disadvantage that there does not exist a method or system that is able to process EMR-datasets automatically.

At least one embodiment of the present invention improves upon the known methods for automatically making predictions based on electronical medical records.

Embodiments of the present invention is directed to a method for creating predictive models for an automated clinical decision support system for automated supervised and semi-supervised classification and treatment optimization of clinical events using EMR data; a prediction-unit for creating prediction-data for an automated clinical decision support system; a method for automated clinical decision support; a Clinical Decision Support System; and a Data processing system.

In the following, embodiments of the invention may be described using examples with respect to predicting the probability of flares of Rheumatoid Arthritis, but the invention is not limited to this application. Embodiments of the invention and embodiments thereof can be used in particular for predicting the future status of a patient having a certain disease, in particular an auto-immune disease.

The method of at least one embodiment of the invention for creating predictive models for an automated clinical decision support system for automated supervised and semi-supervised classification and treatment optimization of clinical events using EMR data is especially applicable (or even designed) for evaluating disease activity in autoimmune diseases or hospital readmiss-ions.

The method of at least one embodiment comprises:
providing a number of EMR-datasets comprising measurements and patient related data of a number of follow-ups,
   if necessary: treating the EMR-datasets in order to estimate missing values and/or to correct outliers and/or to model temporality of measurements
   optionally: forming subgroups of related follow ups within the EMR-datasets,
   providing a target-variable or extract a target-variable from the EMR-dataset,
   providing a number of untrained predictive models which can output probabilities and assign weights to EMR-data of the EMR-datasets, wherein each predictive model is capable of being trained with data using methods of machine learning,
   providing a number of different time horizons,
   performing a nested cross-validation for each time horizon and for each predictive model, and
   selecting a predictive model for each time horizon based on the nested cross-validation.

A prediction-unit of at least one embodiment of the invention for creating prediction-data for an automated clinical decision support system for an embodiment of a method for automated clinical decision support for automated supervised and semi-supervised classification and treatment optimization of clinical events using EMR data comprises a number of trained prediction models for different time horizons (especially one for each different time horizon) as created by at least one embodiment of the above method.

A method of the invention at least one embodiment of for automated clinical decision support for automated supervised and semi-supervised classification and treatment optimization of clinical events using (new) EMR data is explained in the following. In practice, actual EMR-datasets from a follow-up are used, where there are normally no targets (labels) created yet. For example, in a case the label would indicate that a certain event occurs X month after the follow-up, this label cannot be available to the present time of the follow-up, since it will only be known in future.

The method of at least one embodiment comprises:
providing a number of trained prediction models (PM) for a number of time horizons preferably trained by a method of at least one embodiment,
providing an EMR-dataset of a patient comprising measurements and patient related data of a number of follow-ups including the data of a present patient follow-up,
optional: treating the EMR-dataset in order to estimate missing values and/or to correct outliers and/or to model temporality of measurements,
optional: form subgroups of related data given at different follow ups within the EMR-dataset and extract cluster features,
optional: reducing the number of data dimensions in the EMR-dataset and visualizing data of the EMR-dataset in reduced number of dimensions,
calculating the probability of a clinical event in all relevant time horizons, with the number of trained prediction models.

A Clinical Decision Support System of at least one embodiment of the invention for automated supervised and semi-supervised classification and treatment optimization of clinical events using EMR data, comprises a prediction-unit of at least one embodiment of the invention. The Decision Support System is designed to execute a method of at least one embodiment of the invention for automated clinical decision support for automated supervised and semi-supervised classification and treatment optimization of clinical events using EMR data and to visualize the calculated results.

A data processing system of at least one embodiment of the invention, that is especially a computer network system, comprises a data-network, a number of client computers and a service computer-system, wherein the service computer system comprises a Clinical Decision Support System of at least one embodiment of the invention for automated supervised and semi-supervised classification and treatment optimization of clinical events using EMR Data.

At least one embodiment of the invention is also achieved by a computer program product with a computer program that is directly loadable into the memory of a computing system and which comprises program units to perform at least one embodiment of the inventive method when the program is executed by the computing system. In addition to the computer program, such a computer program product can also comprise further parts such as documentation and/or additional components, also hardware components such as a hardware key (dongle etc.) to facilitate access to the software.

A computer readable medium such as a memory stick, a hard-disk or other transportable or permanently-installed carrier can serve to transport and/or to store the executable parts of the computer program product so that these can be read from a processor unit of a computing system. A processor unit can comprise one or more microprocessors or their equivalents.

Particularly advantageous embodiments and features of the invention are given by the claims, as revealed in the following description. Features of different claim categories may be combined as appropriate to give further embodiments not described herein.

### BRIEF DESCRIPTION OF THE DRAWINGS

Other objects and features of the present invention will become apparent from the following detailed descriptions considered in conjunction with the described embodiments and accompanying drawings. It is to be understood, however, that the drawings are designed solely for the purposes of illustration and not as a definition of the limits of the invention.

FIG. 1 displays the binarization approach for categorical and numerical variables.

FIG. 2 displays various weighting functions in a sliding window.

FIG. 3 displays the unsupervised predictive maintenance approach of jet engines based on the PCA.

FIG. 4 displays the k-fold cross-validation for k=10.

FIG. 5 displays the nested cross-validation. In this example, the outer loop has k=5 folds while the inner loop has k=2 folds.

FIG. 6 displays the sliding weighting function for estimating missing values and for accounting for temporality in the EMR laboratory measurements.

FIG. 7 displays the binary encoding step performed after normalized temporal aggregation.

FIG. 8 displays the proposed approach for deriving cluster features from the EMR data.

FIG. 9 displays a proposed technical feature.

FIG. 10 displays the CPLE framework of a preferred example.

FIG. 11 displays the first iteration of the outer loop of the nested cross-validation applied to the labeled EMR data.

FIG. 12 displays the outer loop of a nested cross-validation.

FIG. 13 displays the inner loop of a nested cross-validation.

FIG. 14 displays the outer loop of a nested cross-validation for another iteration.

FIG. 15 displays the outer loop of a nested cross-validation for another iteration.

FIG. 16 displays the first iteration of the outer loop of the nested cross-validation which incorporates the CPLE framework for making use of the unlabeled EMR data.

FIG. 17 displays visualized predicted survival probabilities (in general these could be event-free probabilities for a custom clinical event such as RA flare or hospital readmission) within a time horizon of one year for a patient, given some medication and dose decided by a clinician.

FIG. 18 displays the predicted RA flare probabilities (generalizable to any clinical event such as e.g. death) for an RA patient for five different time horizons computed at one patient visit for a given treatment.

FIG. 19 displays recommendation system based on the multi-objective optimization.

FIG. 20 displays the ranking procedure based on the SVM-Rank algorithm.

FIG. 21 displays a system and/or a method for automated semi-supervised classification and treatment optimization of clinical events using EMR data in the training phase.

FIG. 22 displays a system and/or a method for automated semi-supervised classification and treatment optimization of clinical events using EMR data in the productive or application phase.

FIG. 23 displays a preferred method for creating predictive models for an automated clinical decision support system for automated supervised and semi-supervised classification and treatment optimization of clinical events using EMR data.

FIG. 24 displays a preferred method for automated clinical decision support for automated supervised and semi-supervised classification and treatment optimization of clinical events using EMR data.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. The present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without subdividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Bluray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or porcessors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (procesor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Nonlimiting examples of the non-transitory computer-readable medium include, but are not limited to, rewriteable nonvolatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable nonvolatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

The method of at least one embodiment of the invention for creating predictive models for an automated clinical decision support system for automated supervised and semi-supervised classification and treatment optimization of clinical events using EMR data is especially applicable (or even designed) for evaluating disease activity in autoimmune diseases or hospital readmiss-ions. The method of at least one embodiment comprises:
providing a number of EMR-datasets (EL,EU) comprising measurements and patient related data of a number of follow-ups,
   if necessary: treating the EMR-datasets (EL,EU) in order to estimate missing values and/or to correct outliers and/or to model temporality of measurements
   optionally: forming subgroups of related follow ups within the EMR-datasets (EL,EU),
   providing a target-variable or extract a target-variable from the EMR-dataset (EL,EU),
   providing a number of untrained predictive models (PM) which can output probabilities and assign weights to EMR-data of the EMR-datasets (EL,EU), wherein each predictive model (PM) is capable of being trained with data using methods of machine learning,
   providing a number of different time horizons,
   performing a nested cross-validation for each time horizon and for each predictive model (PM), and
      selecting a predictive model (PM) for each time horizon based on the nested cross-validation.

The method of at least one embodiment comprises:
Providing EMR-datasets.

A (vast) number of EMR-datasets comprising measurements and patient related data of a number of follow-ups is provided. It should be noted although EMR datasets may comprise only measurements, usually not all data collected during follow-ups are measurements. Some data are e.g. patient self-assessment score or demographic data like gender and postal code.
Treating EMR-datasets (preferred).

The EMR-datasets are treated in order to estimate missing values and/or to correct outliers and/or to model temporality of measurements. Preferably, new values are calculated (especially in or with moving windows) simulating missing values or replacing existing values. The modeling of temporality is advantageous to render different datasets with different time-scales in a way that they are comparable.
Forming subgroups (preferred).

Subgroups of related follow ups are formed within the EMR-datasets, in order to extract special features from the data. This forming of subgroups (or “clusters”) is explained further below.
Providing a target-variable (preferred, however necessary for many applications).

This target-variable can be provided manually or extracted directly from the EMR-dataset. The expression “target variable” (also called “output variable” or (mostly in statistics) “dependent variable” or “outcome variable”) is well known in the technical field of KI for a variable that should be estimated.

The provision of the target variable is very advantageous and partly a necessity for a number of preferred embodiments explained below. In the case the predictive model is not implicitly aligned to a target, the target could be provided explicitly by labeling EMR-datasets. The target could be a label assigned to a group of EMR-datasets, e.g. “is there the possibility of a disadvantageous event occurring to the patient”. The provision or extraction of the target variable should be performed whenever possible.

In the case, the target could be extracted from the EMR-datasets they could be referred to as “labeled EMR-datasets”. In the case, the target could not directly be extracted from the EMR-datasets they could be referred to as “unlabeled EMR-datasets”.
Providing predictive models

A number of untrained predictive models are provided (especially chosen from a predefined set of models, including e.g. RandomForest, AdaBoost, Logistic Regression etc.). The predictive models are able to output probabilities and assign weights to EMR-data, wherein each model is capable of being trained with data using methods of machine learning.
Providing time horizons.

A number of different time horizons (e.g. 1 month, 3 months, etc.) are provided. It is one goal of the invention, to create one final best model for each time horizon.
Performing a nested cross-validation for each time horizon and for each predictive model.
Selecting a predictive model for a time horizon based on the nested cross-validation.

A prediction-unit of at least one embodiment of the invention for creating prediction-data for an automated clinical decision support system for an embodiment of a method for automated clinical decision support for automated supervised and semi-supervised classification and treatment optimization of clinical events using EMR data comprises a number of trained prediction models for different time horizons (especially one for each different time horizon) as created by at least one embodiment of the above method.

A method of the invention at least one embodiment of for automated clinical decision support for automated supervised and semi-supervised classification and treatment optimization of clinical events using (new) EMR data is explained in the following. In practice, actual EMR-datasets from a follow-up are used, where there are normally no targets (labels) created yet. For example, in a case the label would indicate that a certain event occurs X month after the follow-up, this label cannot be available to the present time of the follow-up, since it will only be known in future. The Method comprises:
Providing trained prediction models for a number of time horizons, especially trained by the above method.

Especially a prediction unit of an embodiment of the invention can be used. There is especially one single prediction model for each time horizon.
Providing an (especially unlabeled) EMR-dataset of a patient comprising measurements and patient related data of a number of follow-ups including the data of a present patient follow-up.
(Preferred:) Treating the EMR-datasets in order to estimate missing values and/or to correct outliers and/or to model temporality of measurements. This treatment is very advantageous, however only if the EMR-datasets are not flawless. Nevertheless, even if there are flawless EMR-datasets, the method is preferably designed to apply this step, for the case there are EMR-datasets that are not flawless.
(Preferred:) Form subgroups of related data given at different follow-ups within the EMR-datasets and extract cluster (subgroup) features. If used in the model training, clusters (subgroups) are especially provided that are derived in the training phase to extract cluster features.
(Preferred:) Reduce the number of data dimensions in the EMR-datasets and visualizing the EMR-datasets in reduced number of dimensions. Especially visualizing data of the EMR-dataset in reduced number of dimensions derived in the training phase.
Calculate the probability of a clinical event in all relevant time horizons with the number of trained prediction models. This is done especially for different treatment options, e.g. for different medication and/or doses.

In addition, the calculated probability of a clinical event in all relevant time horizons could be displayed or otherwise provided for a user or a using system.

A Clinical Decision Support System of at least one embodiment of the invention for automated supervised and semi-supervised classification and treatment optimization of clinical events using EMR data, comprises a prediction-unit of at least one embodiment of the invention. The Decision Support System is designed to execute a method of at least one embodiment of the invention for automated clinical decision support for automated supervised and semi-supervised classification and treatment optimization of clinical events using EMR data and to visualize the calculated results.

A data processing system of at least one embodiment of the invention, that is especially a computer network system, comprises a data-network, a number of client computers and a service computer-system, wherein the service computer system comprises a Clinical Decision Support System of at least one embodiment of the invention for automated supervised and semi-supervised classification and treatment optimization of clinical events using EMR Data.

The units or modules of embodiments of the invention mentioned above can be completely or partially realised as software modules running on a processor of a computing system. A realisation largely in the form of software modules can have the advantage that applications already installed on an existing system can be updated, with relatively little effort, to install and run the methods of the present application.

At least one embodiment of the invention is also achieved by a computer program product with a computer program that is directly loadable into the memory of a computing system and which comprises program units to perform at least one embodiment of the inventive method when the program is executed by the computing system. In addition to the computer program, such a computer program product can also comprise further parts such as documentation and/or additional components, also hardware components such as a hardware key (dongle etc.) to facilitate access to the software.

A computer readable medium such as a memory stick, a hard-disk or other transportable or permanently-installed carrier can serve to transport and/or to store the executable parts of the computer program product so that these can be read from a processor unit of a computing system. A processor unit can comprise one or more microprocessors or their equivalents.

Particularly advantageous embodiments and features of the invention are given by the claims, as revealed in the following description. Features of different claim categories may be combined as appropriate to give further embodiments not described herein.

At least one embodiment of the invention combines solutions to several embodiments of the above mentioned problems (A) to (F). In the following, embodiments of the invention are explained by explicitly describing how problems (A) to (F) are solved by the inventive concept. In addition, preferred solutions are described. Especially, some of the following embodiments could be for themselves or combined with features of other embodiments an individual invention.

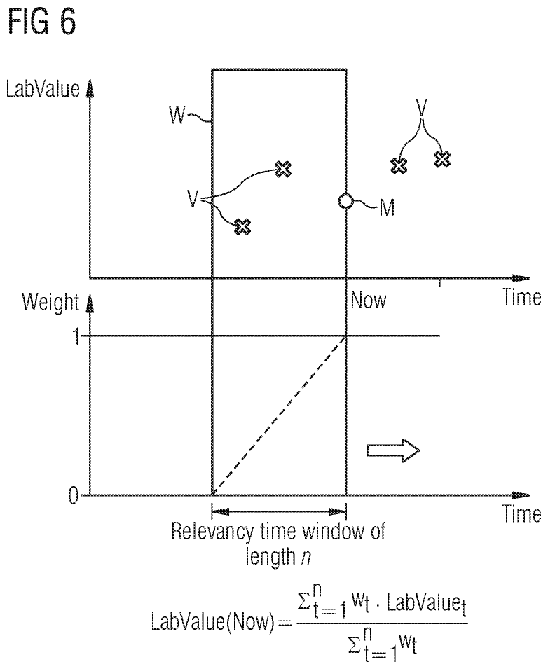

(A) According to an embodiment of the invention, a sliding weighting function is used to estimate missing values in numerical variables. As long as there are some measurements in the relevancy time window, missing values are in particular estimated from them as the sum of weighted past known values. Since such sums can result in values which are not interpretable by clinicians, according to an embodiment of the invention normalization by the sum of weights is used. In this way the estimated values will remain within typical and for clinicians understandable ranges of the corresponding measurements and moreover, the temporality of the measurements is modeled explicitly by value aggregation. In order to fully account for the temporality in the data, known measurements can be replaced by the aggregated ones. This normalized temporal aggregation step is illustrated in FIG. 6.

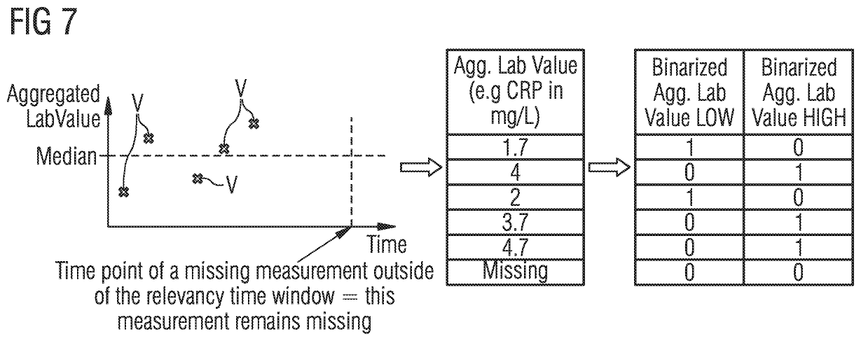

However, if there are no known values within the relevancy time frame, in general such estimation is not possible, i.e. if all previous values within the relevancy time window are missing, the current value will remain missing. Still, with this approach the number of missing values in the EMR is significantly reduced. After performing the normalized temporal aggregation, according to a further embodiment of the invention a binarization approach is applied to encode the remaining missing values as binary zero vectors as described above. Since the number of missing values is already reduced, smaller portion of values will be encoded by dummy binary variables having zero value. This is illustrated in FIG. 7.

In a preferred method, missing values in the EMR-datasets are estimated by using a sliding weighting function, wherein especially missing values are estimated from measurements of the EMR-datasets in a relevancy time window as a sum of weighted past known values, preferably by normalizing this sum by the sum of weights.

Alternatively or additionally, the temporality of measurements in the EMR-datasets is modeled explicitly by value aggregation, wherein in order to fully account for the temporality in the data, known measurements are preferably replaced by the aggregated values.

In a preferred embodiment, after performing one of the afore described steps (especially both steps), in a “normalized temporal aggregation”, a binarization approach is preferably applied to encode the potentially remaining missing values as binary zero vectors.

(B) As described above, detecting and visualizing subgroups within the EMR data using clustering approaches is known in the prior art. To that aim, various well-known clustering algorithms such as e.g. a k-means algorithm are used.

According to a further embodiment of the invention these hidden groups exploited to generate features (i.e. predictors), which might be predictable with respect to the given classification task. In particular, the follow-ups of patients are clustered using some clustering algorithm (e.g. k-means). The optimal number of clusters for the given dataset is determined using known methods, such as the silhouette analysis. Once the hidden groups (i.e. clusters) of patients' follow-ups are obtained, according to a further embodiment of the invention they are used as predictors in the classification of disease activity (i.e. flare prediction, but this is generalizable to other disease data) in the following way: for each follow-up the cluster membership becomes one feature and for each follow-up the Minkowski distance (e.g. Euclidean) to the center of each cluster (called a centroid) becomes an additional feature. In this way, the following number of additional predictors is created: (number of clusters+1).

In a preferred method, the subgroups of related data are formed from different follow-ups within the EMR data by using a clustering algorithm, such as e.g. a k-means algorithm. These subgroups are especially detected and visualized.

In a preferred embodiment, these subgroups are exploited to generate predictors, particularly by clustering follow-ups of patients in the EMR-datasets to subgroups, especially by using some clustering algorithm like k-means. The optimal number of subgroups for a given EMR-dataset is determined, preferably by using silhouette analysis.

The subgroups are used as predictors in the classification of clinical events, e.g. flare prediction in RA, in that for each follow-up the following steps are performed:
A first feature is created from the cluster membership of the follow-up,
Other features are created from the Minkowski distance of the follow-up (e.g. Euclidean) to the center of each subgroup (called a centroid).

For example, if there are three subgroups, a feature (may also be called “Variable” or “Predictor”) defining the distance to the center of each subgroup is generated. In total tree features are generated in this example. In this way, the following number of additional predictors is created: (number of clusters+1).

(C) Patients are data generators just like many technical systems. Therefore, in order to easily monitor changes in their health conditions based on the data collected at their follow-ups, according to a further embodiment of the invention approaches from the area of predictive maintenance of technical systems are used. In particular, the PCA-based condition monitoring approach can be used. Multidimensional EMR patient data is represented in the PCA space using two or maximum three dimensions (so that it can still be visualized and understood by clinicians). All those follow-ups that are associated with the occurrence an event of interest (in the RA use case this is a flare) are represented in the PCA space as points of one color and all other follow-ups as points of another color. These points are likely to fall in two more or less overlapping groups. At each patient visit, when new data is collected, it will be mapped as a point into the PCA space where data of all previous visits of this patient are already marked in the order of appearance. This can be implemented in the animated manner.

In a preferred method, the number of data dimensions in the EMR-datasets is reduced and the EMR-datasets in reduced number of dimensions are visualized. For clarity it is noted that other steps of an embodiment of the inventive method may be performed in the original dimensions.

The number of data dimensions in the EMR-datasets is preferably reduced by using a Principal Component Analysis (“PCA”), especially a PCA-based condition monitoring approach. Patient data of the EMR-datasets (that are often multidimensional) are represented in a PCA-space using two or three dimensions. Data from follow-ups that are associated with a clinical event (in the RA use case this would be a flare) are represented in the PCA-space as points of one individual subgroup. Data from other clinical events form another subgroup (e.g. by color-coding). The subgroups are preferably visualized, especially in a space, where dangerous areas (i.e. areas where there are dangerous conditions for a patient) are marked.

(D) In contrast to applying denoising autoencoders to make use of the unlabeled EMR patient data (the label can be for example disease activity or flare occurrence in rheumatoid arthritis), according to an embodiment of the invention the Contrastive Pessimistic Likelihood Estimation framework (an acronym is “CPLE”) is used. Denoising autoencoders use the unlabeled data only in the pre-training step, to tune the hidden network layer which reduces dimensionality of the EMR data. Then the unlabeled data is discarded and only the labeled data is provided to the trained hidden layer, which reduces its dimensionality and provides the abstract features to a supervised learning algorithm such as the random forest.

Under the CPLE framework, the typical assumptions of the semi-supervised approaches such as the smoothness and the clustering assumption are not required. The CPLE includes the original supervised learning solution (i.e. a classifier trained on the labeled data only) explicitly into the objective function which is optimized, assigning “soft” labels to the unlabeled data. In this way, the potential improvements of the solution are controlled, i.e. the resulting classifier should not perform worse than the one trained on the labeled data only. The amount of improvement depends on the dataset itself as well as on the ratio of the labeled and unlabeled data. Typically, when the number of labeled instances is large, the inclusion of unlabeled instances rarely brings significant improvements and vice versa. With small adjustments, other semi-supervised algorithms (like transductive support vector machines or self-learning classifiers) can be used as well. CPLE can work with any supervised classifier that (a) allows instance weighting and (b) can output probability estimates. The CPLE framework is illustrated in FIG. 10.

A preferred method concerns the case that the EMR-datasets comprise unlabeled data. In this case, the inner loop optimal model of the nested cross-validation is retrained using a Contrastive Pessimistic Likelihood Estimation framework assigning soft labels to unlabeled data. For labeling unlabeled data in the EMR-dataset the following steps are preferably applied:
Create a supervised classifier.

This supervised classifier is created by training and/or tuning based on labeled EMR-data from the EMR-dataset and a predefined grid of hyperparameter values, by using the inner loop of a nested cross validation routine.
Choosing soft-labels.

These soft-labels, (e.g. integer soft-labels) for unlabeled EMR-data are chosen randomly from a given interval of values. One preferred interval is the interval [0,1] for binary classification.
Create a semi-supervised model.

The semi-supervised model (θₛₑₘᵢ) is created by maximizing a CPL-function of the Contrastive Pessimistic Likelihood Estimation framework which includes a supervised model for the chosen soft-labels.
Using the semi-supervised model

The semi-supervised model (θₛₑₘᵢ) is used for updating the randomly chosen soft-labels, in that the CPL-value is maximized.

The steps concerning the CPL-function and the CPL-value are repeated until convergence occurs. Preferably 1000 iterations or more are applied or the steps are preferably repeated until the optimization from one iteration to the next is smaller than 1/1.000.000.

In the following, the basic principle of a CPLE-framework and the above steps are explained. The name Contrastive Pessimistic Likelihood Estimation (CPLE) framework is based on four special embodiments of the framework:

“Contrastive”: The classifier (i.e. the predicted model) that is trained with labeled data only, is used to estimate the optimization by the not-labeled data. The contrast is the contrast between the supervised model vs. the semi-supervised model that are both in the objective function that is optimized.

Pessimistic: The “objective function” that is maximized during the training, is e.g. here a “Log likelihood” or a “generative likelihood” or a “discriminative likelihood”. The soft labels are assigned to the unlabeled data in a way that the improvement of the semi-supervised model with respect to the supervised model is minimal.

Likelihood: The objective function is maximized during the training. The bigger the likelihood, the better the model.

Estimation: The (internal) parameters of the model are estimated.

In an example, the objective Function may be assumed to read:

$$CPL ( θ , {θ}_{sup} \vert X , U ) = {min}_{q {∈}_{K - 1}^{M}} CL ( θ , {θ}_{sup} \vert X , U , q ) \qquad ( 1 )$$

wherein CPL is the “Contrastive Pessimistic Likelihood”—function, the objective function, θ is an unknown semi-supervised model. This model should be found by maximizing the objective function or the parameters for this model for which the function has a maximum, respectively. θₛᵤₚ is a supervised model that is trained with labeled data, X stands for features and labels (data) and U stands for unlabeled data (only features). The soft-labels that should be assigned to the non-labeled data are denoted by q (see e.g. “Contrasive Pessimistic Likelihood Estimation for Semi-Supervised Classification”, M. Loog, IEEE Transactions on pattern Analysis and Machine Intelligence, vol. 38(3), pp 462-475, 2016), the entire contents of which are hereby incorporated herein by reference.

The function CL of above formula can be written as:

CL(θ,θₛᵤₚ|X,U,q)=L(θ|X,U,q)−L(θₛᵤₚ|X,U,q) (2)

wherein L is the log likelihood of the respective model (semi-supervised and supervised, respectively), each dependent on soft-labels q.

The semi-supervised model can then be calculated with the formula:

$${θ}_{semi} = {arg max}_{θ ∈ Θ} CL ( θ , {θ}_{sup} \vert X , U , q ) \qquad ( 3 )$$

A preferred method to accomplish a CPLE-framework comprises the following steps:

a) A supervised model is created, i.e. a model based on labeled data only. This is done by creating a supervised classifier by training and/or tuning based on labeled EMR-data from the EMR-dataset and a predefined grid of hyperparameter values, by using the inner loop of a nested cross validation routine.

b) For data without labels, soft-labels (e.g. integer values) are randomly chosen from a given interval, e.g. the interval [0, 1]. In the case of a binary classification, it is preferred that the values that are >=0.5 are rounded to label 1, lower values are rounded to label 0. In an exemplary case of a rheumatoid arthritis value ‘1’ would indicate an acute attack in a given time period, value ‘0’ would indicate no attack during this period. This is done by choosing (if needed integer) soft-labels for unlabeled EMR-data randomly from a given interval of values (e.g. [0,1] for binary classification);

c) Creating a semi-supervised model with maximizing the objective function (CPL) for the chosen soft-labels. This is done by creating a semi-supervised model θₛₑₘᵢ by maximizing a CPL-function of the Contrastive Pessimistic Likelihood Estimation framework which includes a supervised model trained on the labeled data.

d) Using the semi-supervised model to adjust the randomly chosen soft-labels so that the CPL-value (from above equation) is maximized. This is done by using the semi-supervised model θₛₑₘᵢ for updating the randomly chosen soft-labels, in that the CPL-value is maximized.

e) The steps c) and d) are repeated (i.e. the steps concerning the CPL-function and the CPL-value) until convergence occurs.

(E) As already stated, the nested cross-validation procedure for model selection is designed for supervised learning algorithms. I.e. in each fold (inner or outer) some model is trained in a supervised manner on a training set and evaluated on a test set. This allows the usage of the labeled data only. In addition to the embodiment of the invention of the nested cross-validation to automate the model selection and hyperparameter optimization in modeling the occurrence of clinical events using EMR data, according to another embodiment of the invention it is extended to semi-supervised learning based on the CPLE framework described above.

If only labeled data are to be used, a number of supervised learning algorithms are selected including (but not limited to): logistic regression, linear discriminant analysis, quadratic discriminant analysis, decision tree, random forest, adaboost, gradient boost, bagging classifier, k-nearest neighbor and support vector machine. For each algorithm, a grid of values can be created for its most influential hyperparameters, for example various values of the regularization parameter C are defined for the logistic regression as well as type of the penalty function (L1 vs. L2). Then a nested cross-validation procedure can be performed which builds models for each of those algorithms and for each set of their hyperparameters. At the end, the model with the best nested cross-validation performance measure (typically AUC but can also be F1-score, sensitivity, specificity etc.) can be selected as the best one.

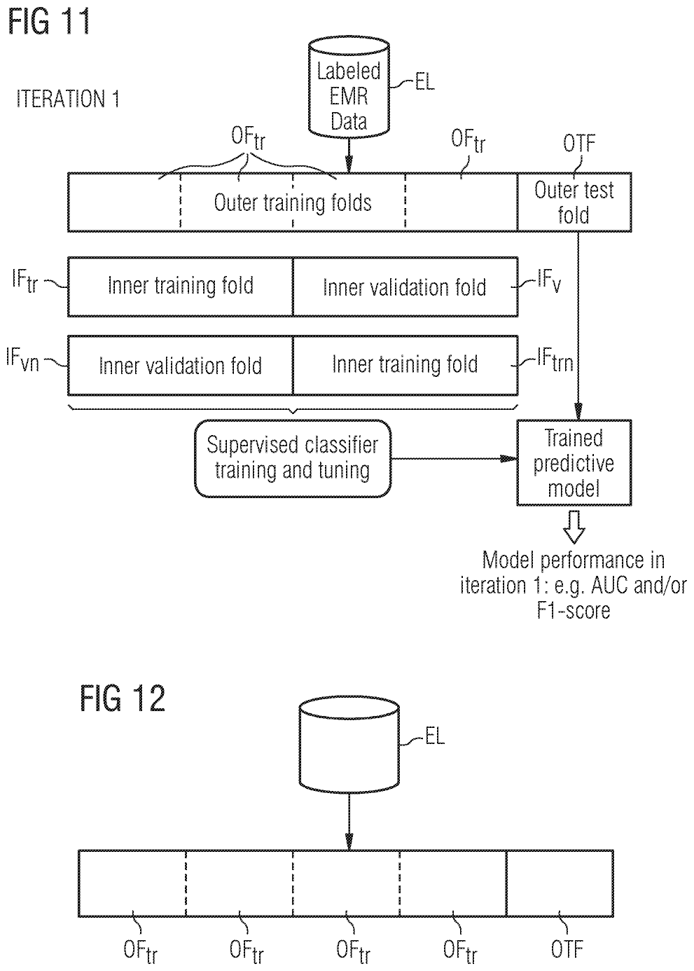

In this way, the model selection process is automated to a large extent (one still needs to define grid of models and their hyperparameter values manually). If all available EMR data includes the label of interest (e.g. RA flares), then this procedure is sufficient to build and select the best model (note: here under the “best” model it is meant the best within the searched scope of potential models). The first iteration of the procedure is illustrated in FIG. 11. The subsequent iterations are conceptually the same with the exception of the different outer and inner training and test/validation folds which changes in each iteration.

However, as described before, in the EMR data the labels are missing for the large portion of instances. Here we propose the semi-automated model selection procedure based on the nested cross-validation for semi-supervised classification. Building on the solution for problem (D) described above, according to an embodiment of the invention the CPLE meta-learner is embedded into the outer loop of the nested cross-validation. When evaluating an algorithm and its hyperparameters, for each fold of the outer nested cross-validation loop a grid search can be performed in the inner loop using labeled training data only to find the best supervised model. Then the CPLE meta-learner is employed receiving the whole dataset (labeled training+unlabeled instances) as well as the best so far obtained model. The CPLE labels the unlabeled instances and retrains the best model, which can then be evaluated on the labeled test set.

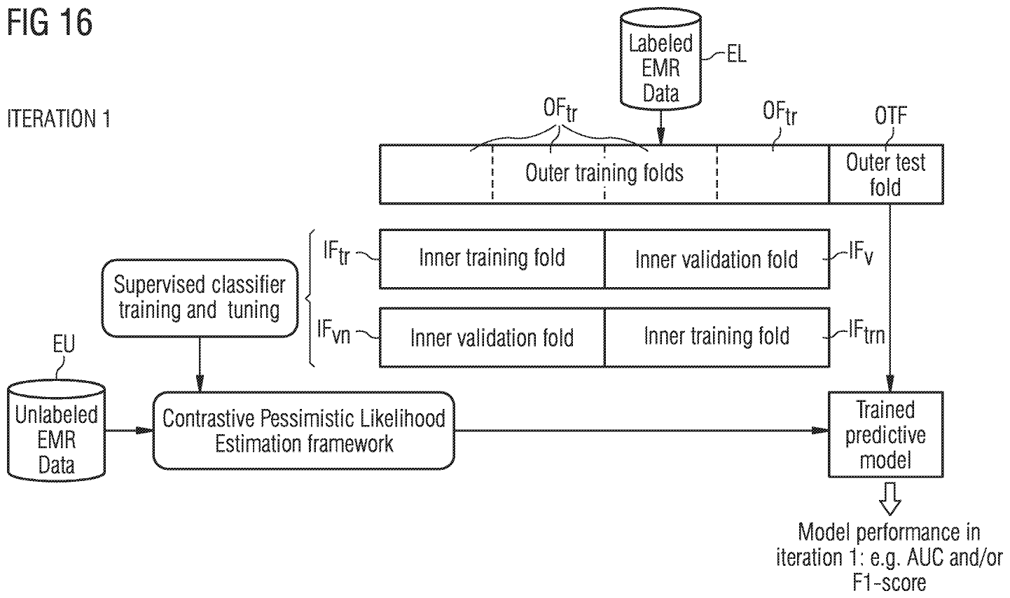

This procedure is performed k times, once for each of the k iteration of the outer nested cross-validation loop. The results can then be aggregated by computing the mean and standard deviation of the selected performance metrics over all outer loop's iterations. The first iteration of the outer loop in the proposed extension is illustrated in FIG. 16. The subsequent iterations are conceptually the same with the exception of the different outer and inner training and test/validation folds which changes in each iteration.

It is important to note that independent of the use of labeled data only or both labeled and unlabeled data, the division to training and test folds is always performed on the group level. The group ID in the EMR data is the patient ID. Such division ensures that all follow-ups (i.e. all data) of a single patient in each of the folds end up either in the training or in the test set. In this way the problem of leakage is avoided, i.e. the model trained in one of the iterations indeed never “saw” any data of patients whose data is used in the test fold of that iteration. In addition to already imposed conservative model performance estimation in the nested cross-validation in comparison to a single cross-validation, this ensures that the estimated performance is further conservative and in the average likely to be slightly better in practice. This is because in the practice, the models will be used to compute predictions for some patients whose past data was used in the model-building process.

For example, each patient has an ID number e.g. 789. All data from this patient collected over years in many follow-ups have the same ID number (789 in this example). The data can be stored in a lot of records, especially for patients with chronic illnesses who need to go to the doctor relatively often. The expression “Group level” refers to this ID number. So every patient represents a group of data. To avoid the phenomenon of “leakage” (which is very negative in machine learning), one should be very cautious when sharing the data in training and testing sets. One has to make sure that all the data in a group (i.e. all patient follow-ups) is converted into either a training set or a test set. Otherwise, if the data is mixed and some patient's FU's in the training set (perhaps future data) and some in the test set, the model already “sees” the patient's future during training. In this case the evaluation of the performance on the test set is not objective. However, by setting this division to two sets at the “group level”, one avoids this subtle problem of model accuracy inflation.

In a preferred method, the predictive models are selected from the group comprising logistic regression, linear discriminant analysis, quadratic discriminant analysis, decision tree, extra tree classifier, random forest, adaboost, gradient boost, bagging classifier, k-nearest neighbor, naïve Bayes classifier and support vector machine.

In a preferred method, for each iteration of the outer loop of the nested cross-validation a set of hyperparameters relevant for the predictive models the following steps are performed. For the sake of clarity, it is noted that each model could have an individual set of hyperparameters. It is preferred to create a grid of values for a number of the most influential hyperparameters of each predictive model. For example, various values of the regularization parameter C are defined for the logistic regression as well as type of the penalty function (L1 vs. L2).

For each predefined set of hyperparameters a predictive model is trained in the inner loop of the nested cross validation using labeled EMR-data of the current outer loop's iteration, followed by the steps:
Select the optimal hyperparameter set for the given model.
Train the inner loop final model using all labeled data of this outer loop's iteration and selected optimal hyperparameters.
Test the inner loop optimal model using labeled test data of this outer loop's iteration.
Aggregate test results of the outer loop iterations, preferably by computing the mean and standard deviation of the selected performance metrics over all outer loop's iterations.
Compare aggregated testing results of the outer loop for different trained predictive models and select the optimal predictive model. The model is selected as the best one with the best nested cross-validation performance measure. Typically, AUC is used, but can also be F1-score, sensitivity, specificity etc.
Train the final predictive model for a (each) time horizon by repeating iterations of the outer loop for each predictive model using all labeled and especially also all unlabeled data.

In a preferred embodiment, for each outer fold of the outer nested cross-validation loop a grid search is performed in the inner loop using labeled training data only to find the best supervised model.

In a preferred embodiment, the best model is retrained and then evaluated on the labeled test set of the outer fold, wherein this procedure is performed a number of times, once for each of the number of iterations of the outer nested cross-validation loop.

This procedure gives as a result the info, which algorithm and with which hyperparameters are best for the given data. However, in one additional step, the final model is created with all instances (all labeled and preferably all unlabeled instances). In addition, it is also important to find the best model and then train it with all the data for each relevant period.

With this method a prognostic model can be generated that computes the probability of an event (e.g. RA flare) occurring within the next X months of current visit (e.g. the target would be defined due to the time horizon of 3 months). This probability can be visualized at each visit, thus showing the doctors an insight into the changes in the risk of the event dependent on their treatment decisions (e.g. medications and a dosage).

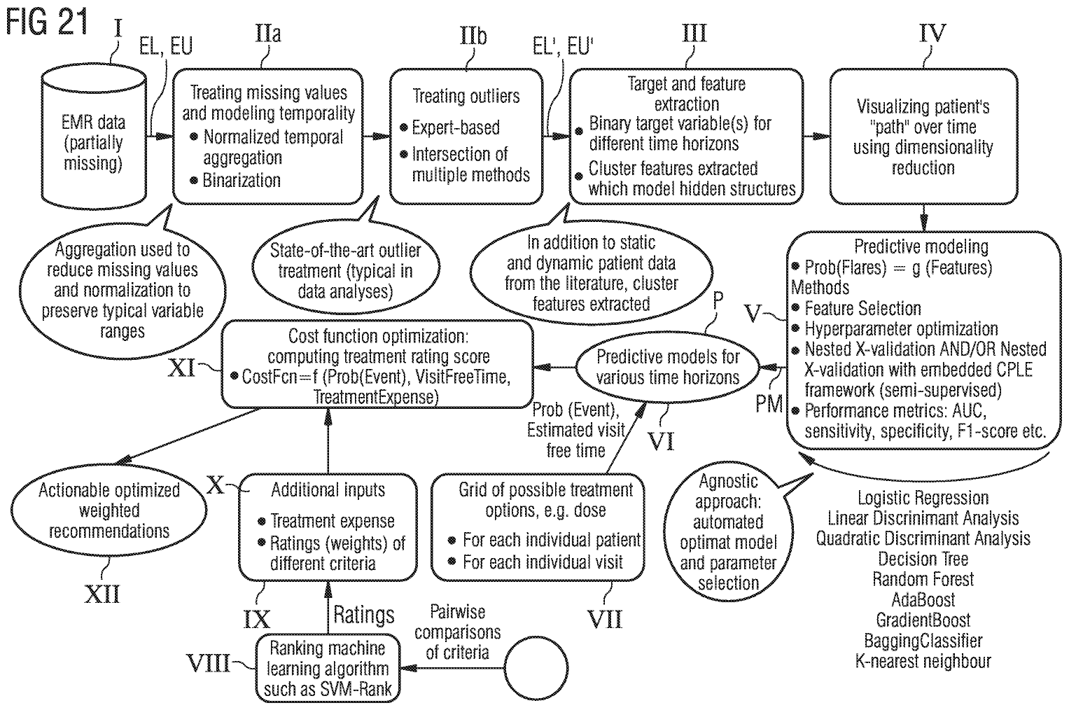

(F) In order to give insights into trade-offs between the risk of an adverse event (e.g. an RA flare, kidney rejection in kidney transplantation, hospital readmission, patient death etc.) over various time horizons (e.g. 1, 3, 6, 9, 12 months from the current follow-up) on the one hand and treatment expenses including indirect expenses caused by frequent patient visits on the other hand, the invention is based according to one of its embodiments on an optimization solution explained in the following. This solution relies on all previously stated solutions for the stated problems. Its core is the trained and conservatively evaluated predictive model which predicts probabilities of an adverse event for a certain time horizon. Assuming a treatment decision is made by a clinician (e.g. a medication is selected with a certain dose level), at each follow-up, as the patient data becomes available, it gets provided to the model which predicts an event probability (e.g. death within a year from the current follow-up). Those predictions can be illustrated for all past follow-ups in a graphic such as the one illustrated in FIG. 21.

The results of (F) are preferably achieved by minimizing a cost function.

This solution can be optimized by the following preferred embodiments.

(F1): Several predictive models are generated, one for each time horizon. For physicians, different time horizons are interesting, instead of only one time horizon of e.g. 3 months. Several prognostic models are generated estimating the probability of an event within e.g. one month, three months, or 6, 9 or 12 months. It has to be noted that these time horizons are disease-specific. These models are summarized in a model called a “meta-model”. This enables a visualization of the probabilities of the event for several time periods.

(F2): Predictions are calculated with the meta-model not only for the current medication or dose but also for a number or plausible drugs or doses. This provides physicians with insight into how the change in medication and dose will affect the risk of clinical events over time.

(F3): Based on these calculated risks, the time in which no follow-up is necessary can be estimated. That has the advantage that time and resources could be saved. In an example where the likelihood of relapses is low (e.g. <50%) within the next 1, 3 and 6 months and >=50% starting from the ninth month, then the physician may make the next visit shortly before the end of the 6th month order. This “visit-free time” could be normalized and quantified on a scale from 0 to 1.

(F4): The costs of medication and dose (which are known to each hospital for each disease) can be normalized (e.g. on a scale from 0 to 1. This has the advantage that the doctor has an overview about the costs, follow-up times and probabilities of the event within different time horizons when there is the need to decide about the appropriate therapy.

(F5): In an example, where physicians have 7 criteria (cost, visit-free time and 5 probabilities of clinical events for 5 time horizons) which they can use to make their decision. However, a recommendation of the decision would be advantageous. To solve this, a number of doctors working in a medical field (e.g. rheumatology) compare the criteria. This could e.g. be accomplished by “pairwise comparisons”. Practically this could be a very short survey, in which a doctor would choose the more important criterion between 2 offered criteria (each pair would be presented to a doctor). The results of this survey would be provided to a ranking algorithm (e.g. SVM Rank) and the algorithm generates the aggregated rating for each criterion. These ratings could be normalized on a scale between 0 to 1 and show aggregated weights of the individual decision criteria for the medical field.

(F6): The weights obtained in the previous step are multiplied by the quantitative values of the individual criteria, forming a “Treatment Rating Score”. Based on this score, the therapy recommendations are given.

(F7): A Decision Support System, which graphically and transparently presents the information about risks for clinical events over various time periods, especially as well as the need for visits and therapy costs. In addition, the rating of therapy decisions is shown for each patient and the three best decisions are suggested.

A preferred method concerning item (F) for automated clinical decision support for Automated Supervised and Semi-supervised Classification and Treatment Optimization of Clinical Events using EMR Data, comprises the additional step calculating and visualizing the probability of a clinical event for different medication and/or doses with the number of trained prediction models for each time horizon.

A preferred alternative or additional method comprises the step estimating the time when no follow-up is necessary based on the calculation(s) of the probability of a clinical event occurring and preferably obtaining the financial costs for a therapy, especially the financial costs for medication and/or doses and/or a follow-up. The financial costs could be derived only once from an information-source. However, since costs could fluctuate over time, it is advantageous to get the actual estimation about the costs.

A preferred Method for automated clinical decision support for Automated Supervised and Semi-supervised Classification and Treatment Optimization of Clinical Events using EMR Data, comprises the additional steps:
Computing ratings of included criteria (e.g. treatment cost, visit-free time, probability of a clinical even for different time horizons) using a ranking algorithm (e.g. SVM-Rank) from pairwise comparisons of those criteria as provided by multiple clinicians. This step could especially be performed only once.
Preferably: Normalize financial costs of various possible treatments (especially medications and/or dosage), preferably in a way that the most expensive treatment (especially medications and/or dosage) correspond to the normalized expense of 1 and the zero dose corresponds to the normalized expense 0. This step could especially be performed only once.
Preferably: Computing 1-normalized visit-free time, wherein preferably normalized visit-free time is normalized to the interval [0,1] in the following way:

a) if the event probability is high (e.g. >=50%) already at the shortest relevant time horizon, then the longest “safe” normalized visit-free time is 0 for the given treatment,

b) if however the predictions for all relevant time horizons are small (e.g. <50%), the longest visit-free period is 1 for the given treatment.
Preferably: Computing a Treatment Rating Score for each plausible treatment as a sum of products of computed ratings for relevant criteria and quantitative values of those criteria (e.g. normalized financial costs and 1-normalized visit-free time as well as an event probability in different time horizons).

Preferably, a recommendation for a suitable treatment based on the Treatment Rating Score is evaluated, wherein e.g. treatments with a smaller score are preferred.

According to a further embodiment of the invention, based on the aforementioned system and method for automated semi-supervised classification of disease activity in autoimmune diseases using EMR data, predictive models for an adverse event of interest can be generated for different time horizons (i.e. a meta-model is generated as a collection of multiple predictive models). This is illustrated in FIG. 18 for the RA flare example.

In the following, the presented embodiments of the invention are summarized, and additional possible embodiments are listed.

According to a further embodiment of the invention, missing values in the electronical medical record are determined based one past values using sliding windows with monotonically decreasing weighting functions, in particular using temporal aggregation. The inventor recognized that by using this approach the amount of missing values in numerical variables can be significantly reduced. Furthermore, the temporal context of the measurements is taken into account by giving highest weight to the most recent and lowest weight to the latest relevant value.

According to a further embodiment of the invention normalization is based on the summed products of variable values and their corresponding weights. The inventor has recognized that using normalization based on summed products the estimated values are kept in realistic and interpretable scales.

According to a further embodiment of the invention a binarization is used to encode the numerical values obtained based on normalized temporal aggregation. The inventor has recognized that in case some missing values remained after performing a normalized temporal aggregation, they can efficiently be modeled as binary vectors using binarization, so that no EMR data gets wasted and every single instance can be used for modeling.

The inventor furthermore recognized that these embodiments enable a direct application of the predictive modeling statistical and machine learning algorithms without wasting any EMR data due to the missing values in input variables. Methods according to these embodiments of the invention can be used for clinical decision support based on the EMR data. Although motivated by the problems observed in the concrete RA use case, the applicability of the proposed approach is independent of the use case and scales to working with the EMR data in general.

Hidden structures in a highly dimensional EMR data often carry a predictive power that is not explicitly utilized by using the original features only for prediction in classification tasks, such as the task of predicting RA flares. According to a further embodiment of the invention the EMR follow-ups are clustered before additional features are derived from clusters (distances to cluster centroids and a cluster label), in particular, these hidden structures can be taken into account explicitly when building classifiers of a disease activity. The inventor recognized that since such features can carry a significant predictive power, the predictive models that use them can have better performance making corresponding prediction algorithms more reliable and accurate.

According to a further embodiment of the invention, the patient follow-up history including the latest data in the reduced PCA space is animated. The inventor recognized that this animation or visualization enables an easy visualization of disease changes over time and shows a patient's path through a disease. This gives clinicians a clear image and intuition about the disease stability for a given patient over time. Furthermore, since follow-ups associated with an event of interest and those where such event was absent (e.g. RA flare) are statically shown in the background, a clinician can not only see how much the disease activity changes over follow-ups for an individual patient, but also judge if the data collected over time and at the current follow-up are in/closer to the group of “dangerous” or “safe” follow-ups. Based on these proximities, appropriate alarms/warnings can be triggered. Furthermore, such visual assets could prove to be valuable for physicians and would significantly contribute to the acceptance of the digital products based on advanced predictive data analytics.

According to a further embodiment of the invention, the CPLE framework is applied for labeling the unlabeled EMR data. In particular, for RA flare occurrence within a given time horizon for the follow-ups that lack this information in the model training phase is estimated. The inventor recognized, that first, the CPLE receives a classifier already trained on the labeled data and tries to improve it using the unlabeled data (opposite of the denoising autoencoders, which performs the training on the labeled data afterwards). In the process of training the CPLE, unlabeled instances from the EMR are labeled and used with originally labeled instances together explicitly to make the final model. A supervised classifier applied after training denoising autoencoders in the state of the art is still restricted to using the same (often small) amount of originally labeled data found in the EMR.

Furthermore, the original features are preserved, i.e. the patient demographic data, blood laboratory measurements etc. are provided to the supervised classifier (e.g. which predicts the RA flares) and clinicians can see and understand what are its predictions based on and how relevant are certain variables for the prediction. As an additional advantage, if a white-box supervised learning algorithm such as the logistic regression is used, a full insight into the model structure is achieved, i.e. it can be easily seen which EMR variables, how and how much are associated with computed predictions. In contrast, denoising autoencoders provide reduced, abstracted features of the hidden layer to the supervised classifier making it fully black-box and not transparent for clinicians (even if white-box models are used). In particular, these advantages can be used for building more transparent clinical decision support systems, which are likely to have higher acceptancy by clients.

According to another embodiment of the invention, nested cross-validation is used to automatically evaluate a number of possible machine learning models and their hyperparameters in the task of predicting the occurrence of an adverse event from the EMR data. The restriction is to use the labeled EMR data only. The inventor recognized that by this embodiment a large number of possible models for predicting disease activity can be evaluated in an automated manner significantly saving time and resources. Furthermore, no assumptions regarding the model structure and optimal decision boundaries between the classes have to be made, so that the evaluation is done in an agnostic manner selecting the solution which maximizes the classification performance.

According to a further possible embodiment of the invention, the semi-supervised CPLE learning framework is embedded into the nested cross-validation for using both labeled and unlabeled EMR data. The inventor recognized that automated model selection using also unlabeled data is enable, which typically dominates EMRs, i.e. much larger datasets can be used in the analysis. Furthermore, by relying on the CPLE semi-supervised learning framework, it is unlikely that the solution obtained in this way will be worse than the solution which relies on labeled data only.

According to a further embodiment of the invention, the nested cross-validation is capable of automatically detecting if provided EMR data also contains unlabeled instances or not. If this is the case, a method according to FIG. 12 or FIG. 16 will be executed; otherwise a method according to FIG. 11 will be executed. The inventor recognized that when modeling the EMR data, the user will not have to differentiate between labeled and unlabeled data, and that the appropriate procedure will be selected automatically which saves the modeling time and costs.

According to a further embodiment of the invention, in possible cases where using labeled data alone provides better predictive performance than adding unlabeled data (such “unusual” cases are known in the literature), the proposed procedure will still select the best model. In particular, modeling with labeled data only vs. using unlabeled data as well is performed automatically and the best model is returned. The inventors recognized that the analyst does not have to perform separate analyses for these two cases and compare the results, which additionally saves time and money.

According to a further embodiment of the invention, a meta-model is proposed, which comprises of models for predicting a probability of an adverse event for different time horizons. The inventor recognized that by this embodiment the user (clinician) gets a deeper insight into the effects of his/her prescribed treatment (e.g. medications and dose) over time, in particular for different time horizons.

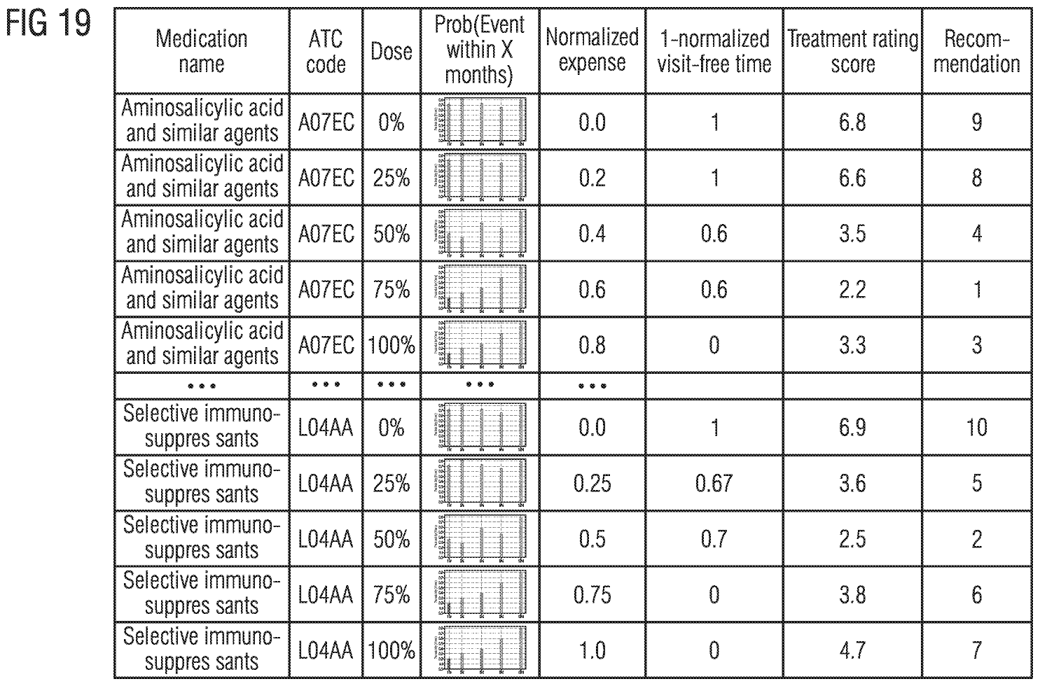

According to a further embodiment of the invention, the previous embodiment is provided not only for the currently prescribed treatment but also for all feasible treatments. The inventor recognized that a “what if” analysis enables to simulate the meta-model with various possible treatment decisions defined in a grid (e.g. various medications and their dose). In particular, it is possible to provide their overview together with graphically represented effects of the corresponding treatment decisions on the disease activity over different time horizons to the user (as illustrated in FIG. 19, columns 1-4).

According to a further embodiment of the invention, a possible visit-free time (i.e. the time in which no follow-up is required since the predicted risk of an adverse event is low) is estimated based on the predicted event probability for different time horizons. The inventor recognized that based on this embodiment a clinician can plan patient follow-ups based on the predicted future disease activity and make better schedules saving time.

According to another embodiment of the invention, both estimated visit-free time and treatment expense are used for building a treatment recommendation system in addition to estimated event risk for different treatments. In particular, treatment expenses (e.g. monetary cost of drugs) are known to clinicians/their institutions. The inventors recognized that such a recommendation system takes not only different event risks into the account, but also the available resources at the hospital, like clinician's time and available budget.

According to a further embodiment of the invention, the computing of treatment recommendations is based on a rating of relevant criteria based on the aggregated feedback (pairwise comparisons) provided by clinicians at the corresponding department. The inventors recognized that by this embodiment a uniform rating is established for the whole department for various optimization criteria (e.g. expense vs. event risk for the horizon of X months) using algorithms such as SVM-Rank. In this way, the variation in treatment approaches between different clinicians is modeled and enables better computation of the treatment recommendation score.

According to a further embodiment of the invention, a treatment recommendation score is calculated taking into account various criteria (different risk probabilities, visit-free time as well as the treatment expense) which are weighted according to their importance to clinicians. The inventor recognized that this score can be used to rank treatment recommendations and propose the best one (or a couple of the best ones) to a clinician.

According to a further embodiment of the invention, a clinical decision support system is created in almost an automated manner based on several or all embodiments of the presented invention. The inventor recognized that by this embodiment the modelers task is simplified and he/she can focus more on understanding the clinical questions and their impact on models and not that much on the modeling itself. Furthermore, the clinicians profit from the transparency of models included in the meta-model as well as from the transparency of the recommendation system.

In the previous parts the invention was described in terms of a method. Furthermore, embodiments of the invention are also directed to a data processing system configured for executing any of the described methods. In particular, the data processing system can comprise a calculation unit, a memory unit and/or an interface.

Such a data processing system (or Computer network system) can, for example, comprise a cloud-computing system, a computer network, a computer, a tablet computer, a smartphone or the like. The data processing system can comprise hardware and/or software. The hardware can be, for example, a processor system, a memory system and combinations thereof. The hardware can be configurable by the software and/or be operable by the software.

The data processing system may be a (personal) computer, a workstation, a virtual machine running on host hardware, a microcontroller, or an integrated circuit. As an alternative, the data processing system can be a real or a virtual group of computers (the technical term for a real group of computers is “cluster”, the technical term for a virtual group of computers is “cloud”).

An interface can be embodies as a hardware interface or as a software interface (e.g. PCI-Bus, USB or Firewire). In general, a calculation unit can comprise hardware elements and software elements, for example a microprocessor, a field programmable gate array (an acronym is “FPGA”) or an application specific integrated circuit (an acronym is “ASIC”). A storage unit (another name is “memory unit”) can be embodied as non-permanent main memory (e.g. random access memory) or as permanent mass storage (e.g. hard disk, USB stick, SD card, solid state disk). The input/output unit can comprise means for inputting data (e.g. a keyboard or a mouse) and/or means for outputting data (e.g. a display or a printer).

In another embodiment, the invention relates to a computer program product comprising a computer program, the computer program being loadable into a memory unit of a data processing system, including program code sections to make the data processing system execute a method according to an embodiment of the invention when the computer program is executed in the data accessing system.

In another embodiment, the invention relates to a computer-readable medium, on which program code sections of a computer program are saved, the program code sections being loadable into and/or executable in a data processing system to make the data processing system execute one of the embodiments of the invention when the program code sections are executed in the data processing system.

The realization one of the embodiments of the invention by a computer program product and/or a computer-readable medium has the advantage that already existing providing systems can be easily adopted by software updates in order to work as proposed by one of the embodiments of the invention.

The computer program product can be, for example, a computer program or comprise another element apart from the computer program. This other element can be hardware, for example a memory device, on which the computer program is stored, a hardware key for using the computer program and the like, and/or software, for example a documentation or a software key for using the computer program.

In a preferred embodiment, the nested cross validation is not absolutely necessary. This embodiment pertains to using one or more of the following methods (e.g. in combination) for treating EMR-datasets and/or for obtaining results from EMR-datasets:

a) Estimate missing values in the EMR-datasets by using a sliding weighting function, wherein especially missing values are estimated from measurements of the EMR-datasets in a relevancy time window as a sum of weighted past known values, preferably by normalizing this sum by the sum of weights.

And/or

b) Model the temporality of measurements in the EMR-datasets explicitly by value aggregation, wherein in order to fully account for the temporality in the data, known measurements are preferably replaced by the aggregated values.

And/or

c) Form subgroups of related data from different follow-ups within the EMR datasets by using a clustering algorithm,
wherein the optimal number of subgroups for a given EMR-dataset is determined,
and wherein the subgroups are used as predictors in the classification of clinical events in that for each follow-up
a first feature is created from the cluster membership of the follow-up,
other features are created from the Minkowski distance of the follow-up to the center of each subgroup.

And/or

d) Reduce the number of data dimensions in the EMR-datasets and the EMR-datasets in reduced number of dimensions are visualized.

And/or

e) Perform a nested cross-validation,
wherein for each iteration of the outer loop of the nested cross-validation a set of hyperparameters relevant for the predictive models, wherein it is preferred to create a grid of values for a number of the most influential hyperparameters of each predictive model,
wherein for each predefined set of hyperparameters a predictive model is trained in the inner loop of the nested cross-validation using labeled EMR-data of the current outer loop's iteration,
followed by the steps:
   select the optimal hyperparameter set for the given predictive model,
   train the inner loop final model using all labeled EMR-data of this outer loop's iteration and selected optimal hyperparameters,
   test the inner loop optimal predictive model using labeled test data of this outer loop's iteration,
   aggregate test results of the outer loop iterations, preferably by computing the mean and standard deviation of the selected performance metrics over all outer loop's iterations,
   compare aggregated testing results of the outer loop for different trained predictive models and select the optimal predictive model,
   train the final predictive model for a time horizon by repeating iterations of the outer loop for each time horizon and each predictive model using all labeled EMR-data and all unlabeled EMR-data.

And/or

f) In the case that the EMR-datasets comprise unlabeled EMR-data, retrain the inner loop optimal model of the nested cross-validation using a Contrastive Pessimistic Likelihood Estimation framework assigning soft-labels to unlabeled EMR-data, wherein for labeling unlabeled EMR-data of the EMR-datasets the following steps are preferably applied:
create a Supervised classifier by training and/or tuning based on labeled EMR-data from the EMR-dataset and a predefined grid of hyperparameter values, by using the inner loop of a nested cross validation routine,
choosing soft-labels for unlabeled EMR-data randomly from a given interval of values,
create a semi-supervised model θₛₑₘᵢ by maximizing a CPL-function of the Contrastive Pessimistic Likelihood Estimation framework which includes a supervised model for the chosen soft-labels,
using the semi-supervised model θₛₑₘᵢ for updating the randomly chosen soft-labels, in that the CPL-value is maximized,
repeating the steps concerning the CPL-function and the CPL-value until convergence occurs.

And/or

g) Calculate and especially visualize the probability of a clinical event in all relevant time horizons, with the number of trained prediction models, especially for different medication and/or doses with the number of trained prediction models for each time horizon.

And/or

h) Estimate the time when no follow-up is necessary based on the calculation(s) of the probability of a clinical event occurring.

And/or

i) Obtain the financial costs for a therapy, especially the financial costs for medication and/or doses and/or a follow-up.

And/or

j) Compute ratings of included criteria using a ranking algorithm from pairwise comparisons of those criteria as provided by multiple clinicians, and
preferably normalize financial costs of various possible treatments, preferably in a way that the most expensive treatment correspond to the normalized expense of 1 and the zero dose corresponds to the normalized expense 0, and/or
preferably compute 1-normalized visit-free time, wherein preferably normalized visit-free time is normalized to the interval [0,1] in the following way:
a) if the event probability is high already at the shortest relevant time horizon, then the longest “safe” normalized visit-free time is 0 for the given treatment,
b) if however the predictions for all relevant time horizons are small (e.g. <50%), the longest visit-free period is 1 for the given treatment, and/or
preferably compute a Treatment Rating Score for each plausible treatment as a sum of products of computed ratings for relevant criteria and quantitative values of those criteria.

Wherever not already described explicitly, individual embodiments, or their individual embodiments and features, can be combined or exchanged with one another without limiting or widening the scope of the described invention, whenever such a combination or exchange is meaningful and in the sense of this invention. Especially some features described here could form individual inventions, especially in combination with other features of this description. Advantages which are described with respect to one embodiment of the present invention are, wherever applicable, also advantageous of other embodiments of the present invention.

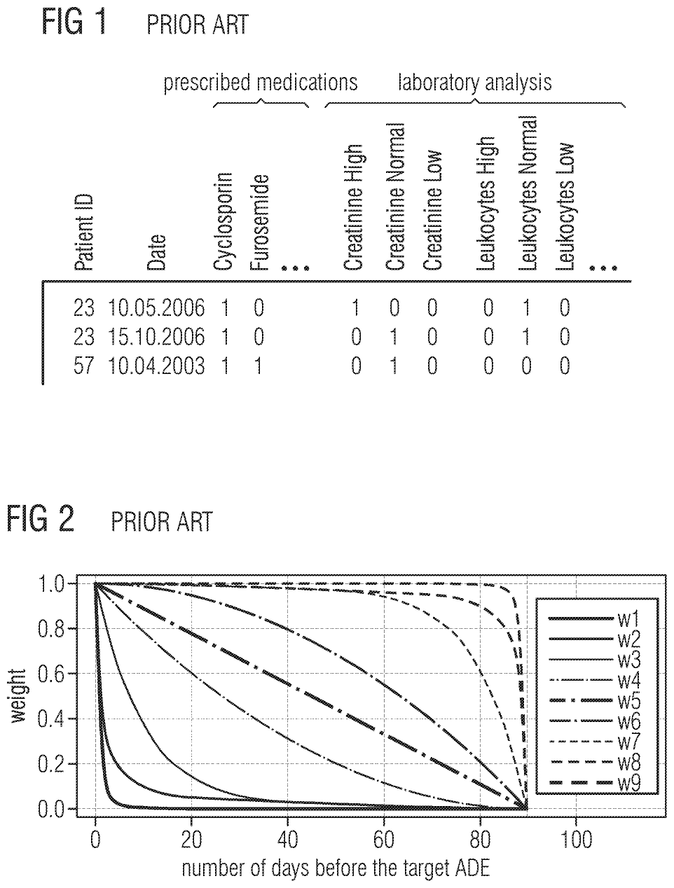

FIG. 1 shows the binarization approach for categorical and numerical variables. In view of a measured value, here e.g. values from measurements of the Creatinine level or the level of leukocytes, and one or more given thresholds (here two thresholds for creating three ranges: low, normal and high), binary variables are created. A ‘1’ is applied if a value lies in one of the intervals given by the thresholds, a ‘0’ is applied if not. In the first line of FIG. 1 the measured Creatinine value is higher than the upper threshold, what results in a ‘1’ in the “Creatinine high” column. In the first and second line of FIG. 1 the measured leucocytes value is between the upper and lower threshold, what results in a ‘1’ in the “Leukocytes Normal” column.

FIG. 2 shows various weighting functions in a sliding window for modeling temporal dependencies of clinical events. The time frame (the length of the sliding window) defined here is about 90 days. In total there are nine weighting functions defined that differ in their decay over the time. The decay of the weighting functions shows the decrease of the relevancy of the past data for the current disease activity the further the point of measurement lies in the past. All weighting functions having their maximum value of 1 at the most recent follow-up recorded in the patient's EMR and its minimum value of 0 at the last time point (90 days) within the relevancy time frame. Weighting function w1 shows a rapid decrease soon after the recent follow up, so that the contribution of previous follow-ups in the past is neglectable, w9 shows only a minimum decay over a long period, so that that the contribution of previous follow-ups in the past is serious, w5 shows a linear decay so that the contribution of previous follow-ups in the past linearly decreases the more time has been elapsed.

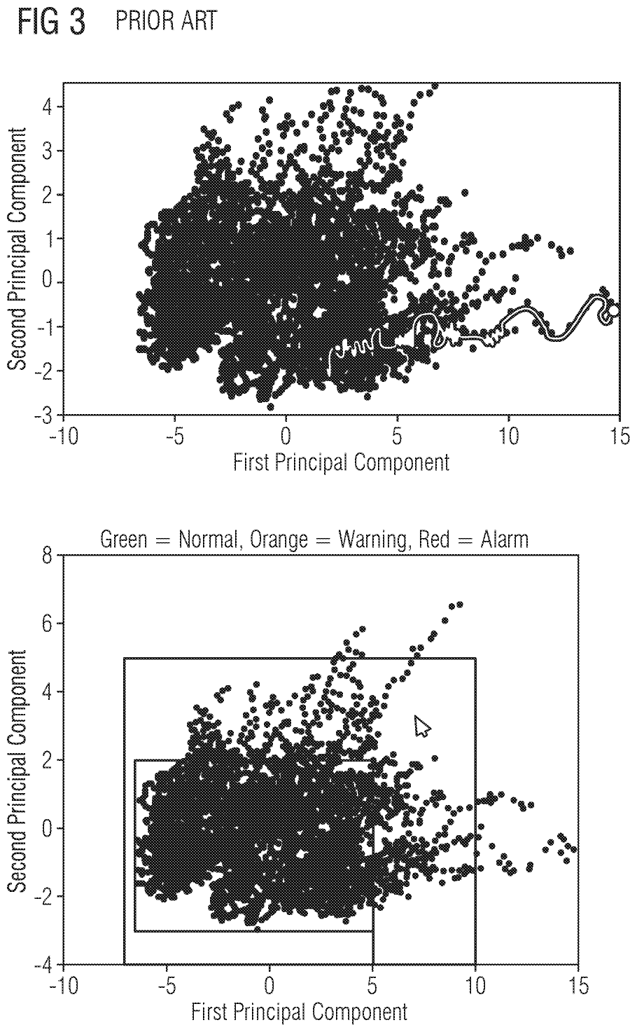

FIG. 3 shows the unsupervised predictive maintenance approach of jet engines based on the PCA. Changes in the health condition of an engine are represented as a path in the PCA space (marked in the upper image). Warnings and alarms are triggered when the path enters the intermediate and the outer zone in the lower picture, respectively.

The PCA space can be received by dimensionality reduce relevant parts of the EMR-data using methods based on Principal Component Analysis (PCA). By uniting corresponding points (representing corresponding measurements of different follow-ups), the static visualization can be used to show changes over time.

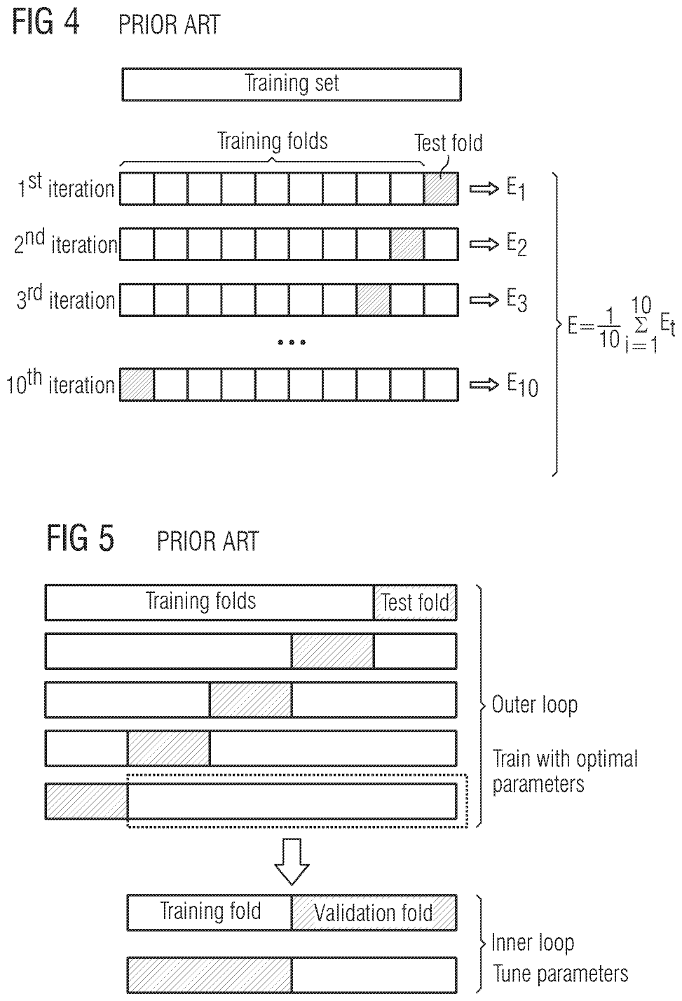

FIG. 4 shows the k-fold cross validation for k=10. In this procedure, the available dataset is divided in 10 mutually exclusive partitions (“folds”), mostly by random sampling without replacement. In the first iteration, 9 folds (“training folds”) are used for training a model which is then evaluated on the remaining fold (“test fold”). This is repeated 10 times, each time choosing a different test fold. In each iteration, some performance metrics is computed, such as the Area Under the receiver operating Characteristics (AUC) or classification error. Afterwards, the mean value and the standard deviation of the performance metrics are computed over the 10 iterations.

FIG. 5 shows the nested cross-validation. In this example, the outer loop has k=5 folds while the inner loop has k=2 folds based on the training folds of the outer loop. The inner loop tunes the hyperparameters and the outer loop estimates the performance.

FIG. 6 displays the sliding weighting (linear) function for estimating missing values M (black dot in the figure; Xs represent known values V) and for accounting for temporality in the EMR laboratory (numerical) measurements. In the figure there are two measurements in the relevancy time window W. Thus, a missing value M (dot) can be estimated as the sum of weighted past known values V. In order to fully account for the temporality in the data, known measurements can, thus, be replaced by the aggregated ones.

However, if there are no known values V within the relevancy time frame, the missing value will remain missing, as shown in FIG. 7.

FIG. 7 displays the binary encoding step performed after normalized temporal aggregation. After performing the normalized temporal aggregation, a binarization approach is applied to encode the remaining missing values as binary zero vectors. This is done e.g. as explained in the example of FIG. 1 by first creating a dataset of values (in this figure a table with five values) and then binarizing these values by comparing them with given thresholds.

The side effect of performing the steps illustrated in FIG. 6 and FIG. 7 is that predictive algorithms gets structures in a tabular format without missing values and a wide range of statistical and machine learning algorithms for predictive modeling can be applied.

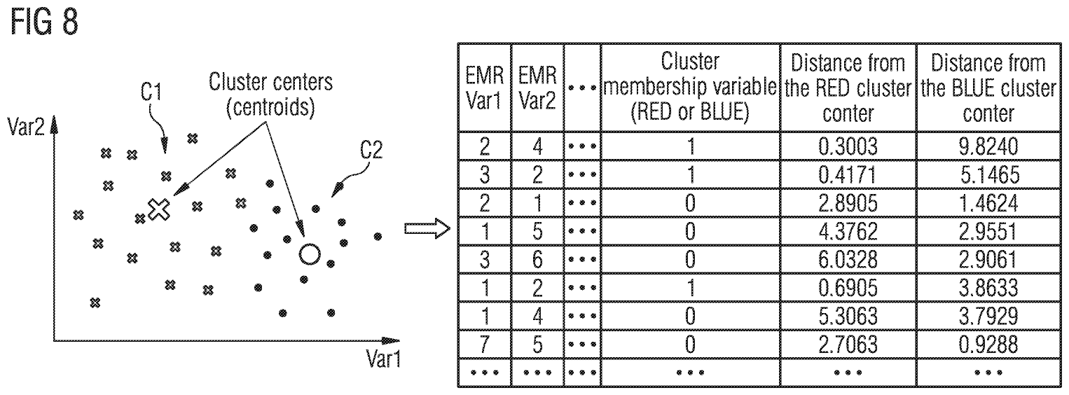

FIG. 8 displays the proposed approach for deriving cluster features from the EMR data. Different clusters C1, C2 are denoted by different symbols in a plane defined by two different parameters Var1, Var2 used for clustering the follow-ups of the EMR-data. Once the clusters C1, C2 of patients' follow-ups are obtained, they are used as predictors in the classification of disease activity (e.g. flare prediction) as described in the following.

For each follow-up the cluster membership becomes one feature (see 4th column of the table) and for each follow-up the Minkowski distance to the center of each cluster (big X and big dot) becomes an additional feature (see columns 5 and 6). In this way, the following number of additional predictors is created: (number of clusters+1). Here the Clustering-Algorithm has found two subgroups (clusters) in the EMR-datasets. The “additional predictors” are the distances from the center of these two clusters (in this case 2 distances for the two clusters), as well as the “membership” concerning the clusters. Therefore, in this example the number of additional predictors derived is 3, what is number of clusters+1 for the membership information. In a simple example, the additional predictors are simply additional variables (e.g. columns in a table) with values that could be used for predictions. In the case there are 3 clusters recognized in the EMR-datasets, there are derived 3 distances from 3 centers plus the membership, this are 4 values in total.

In this figure it is assumed that two clusters C1, C2 are optimal for the given dataset. The Figure is simplified for illustration purposes; the approach generalizes to an arbitrary number of EMR variables and optimal clusters C1, C2.

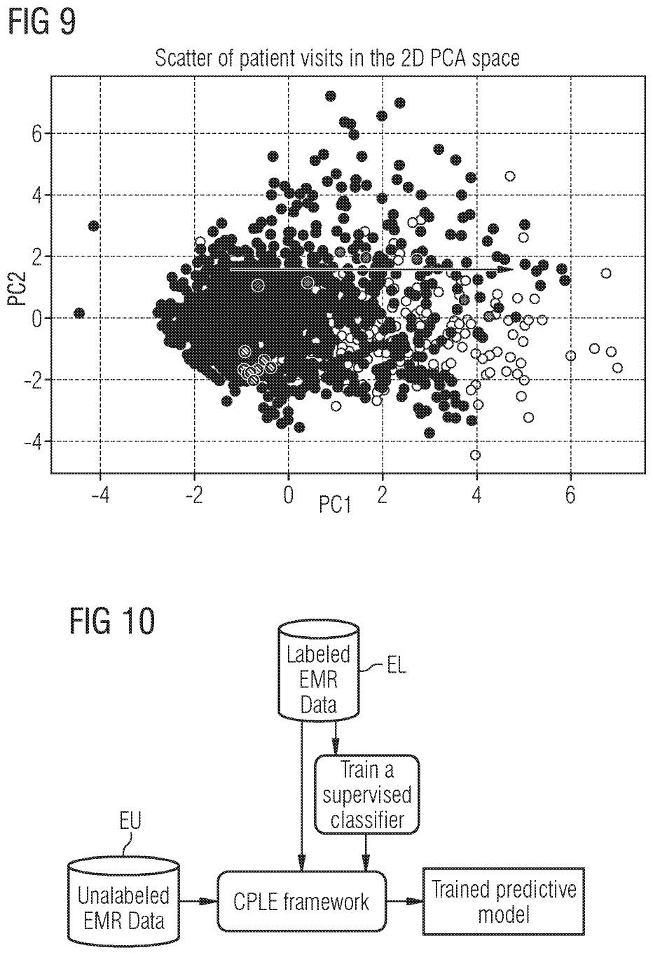

FIG. 9 displays a proposed technical feature. Each dot represents one patient follow-up. The black dots represent follow-ups after which adverse event (e.g. RA flare or even patient death) did not happen within the time horizon of interest (e.g. 3 or 6 months). The white dots are those associated with the occurrence of the adverse event. These are mostly grouped on the right side of the 2D PCA space. The group of light shaded dots on the lower left represent all available follow-ups of a single patient that never experienced an adverse event. One can see that they are all mapped to the left side of the graph, where follow-ups without adverse event are grouped. Moreover, the patient data is stable over multiple follow-ups, i.e. the variance of the PCA values over time is relatively small as the light shaded dots (of this group) fall close to each other. In contrast to this patient, dark shaded dots represent the follow-ups of a patient who eventually did experience an adverse event (last dark shaded dot on the right). The black arrow is added to illustrate the temporal order of those follow-ups (normally, this will be presented to clinicians as animation). The first dark shaded dot on the left (i.e. mapped data of the first follow-up) is relatively deep within the black cluster. Each subsequent follow-up is mapped more to the right, toward the “dangerous” white cluster. Moreover, in contrast to the light shaded dots, the variance of this patient's data is high. Having such (animated) visualization of patient visits can help clinicians to get an impression about the disease activity over time. Warnings and alarms can be triggered when the follow-up data get mapped closer to the “dangerous” cluster of the follow-ups.

FIG. 10 displays the CPLE framework of a preferred example. It uses both labeled EMR-data EL and unlabeled EMR-data EU in the EMR-datasets as well as a supervised classifier trained on the labeled data only. The final trained predictive model expects the inputs in the same form as they are given in the EMR, i.e. no abstract features are created.

The CPLE includes the original supervised learning solution (i.e. a classifier trained on the labeled data only) explicitly into the objective function which is optimized, assigning “soft” labels to the unlabeled data. In this way, the potential improvements of the solution are controlled, i.e. the resulting classifier should not perform worse than the one trained on the labeled data only. The amount of improvement depends on the dataset itself as well as on the ratio of the labeled and unlabeled data. Typically, when the number of labeled instances is large, the inclusion of unlabeled instances rarely brings significant improvements and vice versa. With small adjustments, other semi-supervised algorithms (like transductive support vector machines or self-learning classifiers) can be used as well.

FIG. 11 displays the first iteration of the outer loop of the nested cross-validation applied to the labeled EMR data EL.

The nested cross-validation is explained with the help of FIGS. 12 to 15. The following pseudocode should help to get an overview over the practical process performed by the nested cross validation, wherein the steps 1, 2, 3 and 4 (“For . . . ”) are loops surrounding parts of the code. Loop (1) surrounds all following steps, loop (2) surrounds steps (3) to (9), loop (3) surrounds steps (4) to (8) and loop (4) surrounds step (4a).

(1) For each relevant time horizon (e.g. 1 month, 3 months, etc.; for each horizon one final best model will be created):

(2) For each untrained model which can output probabilities (from the predefined set of models e.g. including RandomForest, AdaBoost, Logistic Regression etc.).

(3) For each iteration of the outer loop of the nested cross-validation.

(4) For each predefined set of hyperparameters (defined in a grid) relevant for the given untrained model.

(4a) Train a model in the inner loop of the nested cross validation using labeled training data of the current outer loop's iteration.

(5) Select the optimal hyperparameter set for the given model.

(6) Train the inner loop final model using all labeled data of this outer loop's iteration and selected optimal hyperparameters.

(7) If unlabeled data available: retrain the inner loop optimal model using the CPLE Framework which assigns soft labels to unlabeled data.

(8) Test the inner loop optimal model using labeled test data of this outer loop's iteration.

(9) Aggregate test results of the step (3) over outer loop iterations.

(10) Compare aggregated testing results of the outer loop for different trained models and select the optimal one.

(11) Train the final model for this time horizon by repeating the steps (4)-(7) for the optimal model using all labeled and all unlabeled data.

FIGS. 12 to 15 show steps (3) to (9) of this pseudocode.

In an example it could be assumed that step (1) starts with the time horizon of 1 month and in step (2) the first model to evaluate is chosen to be Log.-Regression. Now is the task of the steps (3-9) to evaluate various hyperparameter values of Log.-Regression and find the best one (done in the inner loop) and to evaluate the expected performance of the Log.-Regression models trained with optimal hyperparameter values and evaluated on the independent test sets (done in the outer loop).

Iteration 1 of the Outer Cross-Validation

In FIG. 12, step (3) is shown. In the first iteration of the outer cross-validation all labeled EMR data EL are taken and divided into outer training folds OFₜᵣ and one outer test fold OTF (or training and test set of data). The expression “outer” indicates that the folds are situated in the outer loop of the nested cross validation.

Only data from outer training folds OFₜᵣ go into the inner loop of the cross-validation. The Outer test fold OTF is kept as an independent test set for later testing. Outer test data is used later in the step (8).

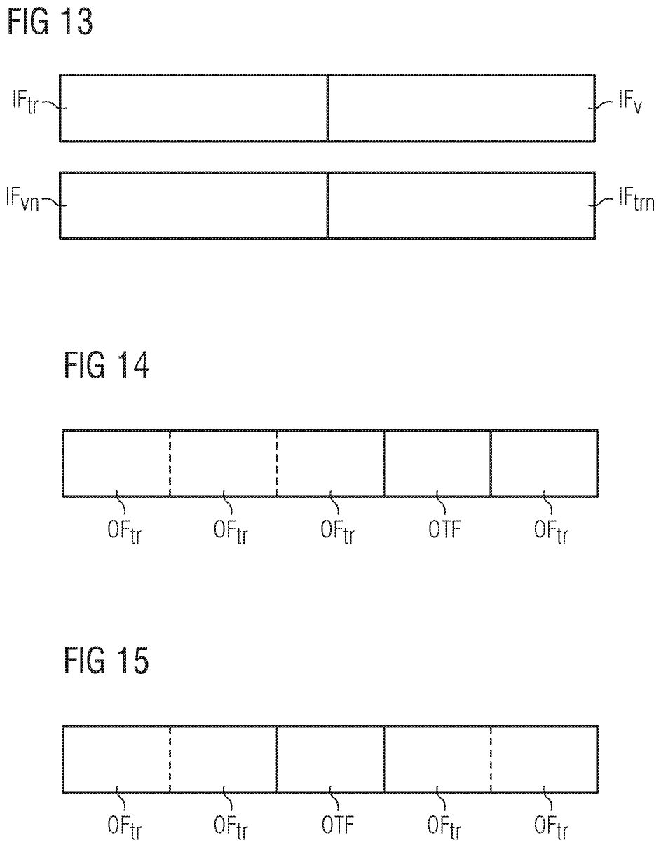

In FIG. 13, step (4) is shown. For the untrained model that we currently want to evaluate (logistic regression as defined in step (2) and time horizon of 1 months as defined in step (1)), a grid of this model's specific hyperparameters is defined (in the case of logistic regression these are the type of regularization (L1 vs. L2) and the value of the regularization parameter C). These are specific for logistic regression, i.e. when some other model will be evaluated e.g. random forest, there will be some other hyperparameters, specific for this other model.

| | | |
|---|---|---|
| Log.-Regression | L1 | 0.01 |
| Log.-Regression | L1 | 0.1 |
| Log.-Regression | L2 | 0.01 |
| Log.-Regression | L2 | 0.1 |

This is the so-called grid of hyperparameters for logistic regression. There may be many more values of C that the modeler (data analyst) wants to investigate but normally a couple of “typical” values are taken due to time constrains.

Now the step (4) says that for each set of hyperparameters (each of the 4 rows in the table above), a logistic regression model will be “assessed” in the inner cross-validation. It should be kept in mind that the inner cross-validation may use only the data from the training folds of the outer cross-validation.

Step (4a) executes this assessment—in each iteration of the inner cross-validation a model is trained on the training set and tested on the test set (of the corresponding inner loop iteration). The figure below shows how 2-fold inner cross validation (there may be more iterations than just 2, this is decided by modeler/data scientist; the more folds, the longer the procedure will execute. In practice one normally takes 5 or 10 iterations).

In the upper left box, a Log.-Regression model with L1 and C=0.01 is trained on this inner training fold IFₜᵣ (data). In the upper right box, a Log.-Regression model with L1 and C=0.01 is validated (tested) on this inner validation fold IFᵥ. Performance of the model is saved for later aggregation.

In the lower right box, again a Log.-Regression model with L1 and C=0.01 is trained but this time on this new inner training fold IFₜᵣₙ(data). This new Log.-Regression model with L1 and C=0.01 is validated (tested) on this new inner validation fold IFᵥₙ. Performance of the new model on this new test data is saved for later aggregation.

After all iterations of the inner loop (i.e. inner cross-validation) are performed, the test results are averaged. Usually a performance metric like accuracy of classification error is logged for each test set and averaged at the end. The output of the step (4a) looks like this:

| | | | |
|---|---|---|---|
| Log.-Regression | L1 | 0.01 | 30 |
| Log.-Regression | L1 | 0.1 | 20 |
| Log.-Regression | L2 | 0.01 | 10 |
| Log.-Regression | L2 | 0.1 | 20 |

Step (5) does the following:

| | | | |
|---|---|---|---|
| Log.-Regression | L1 | 0.01 | 30 |
| Log.-Regression | L1 | 0.1 | 20 |
| Log.-Regression | L2 | 0.01 | 10 |
| Log.-Regression | L2 | 0.1 | 20 |

So in the step (5), the set of hyperparameters for logistic regression model which minimizes the classification error is selected as the optimal parameter set. This is the set with L2 and C=0.01 which had the classification error of 10% (third line).

It should be noted, that the interest is in how this logistic regression model with the optimal values of hyperparameters selected in this way would perform in reality on never-seen data. Its error has in fact been measured in the inner loop (10%), but this error is used for selecting the best hyperparameters and that makes it not suitable for making statements about the expected performance in new, never-seen data. That is the purpose of the outer loop: it always keeps a test set which is independent and not used for selecting the hyperparameters. There will be a later use of this set to measure the expected error in the real-world.

Step (6) now takes all the data of the inner cross-validation (i.e. the training data of the corresponding outer loop iteration) and the optimal hyperparameters (L2 and C=0.01) and trains a logistic regression model on this data using the optimal hyperparameter values.

Step (7) checks if unlabeled data is available and if yes, retrains the model from step (6) using the CPLE framework. The output of the step (7) is a logistic regression model with hyperparameters L2 and C=0.01 trained on all the training data of the corresponding outer loop iteration (all are labeled)+all unlabeled data.

Step (8) finally takes the test set of this iteration of the outer loop (all data in this set are labeled) and measured the performance of the model from step (7) on this test set. The result of the step (8) is performance metric value saved for later averaging of the outer loop results (this metric is e.g. classification error or accuracy, or something called AUC or F1-score etc.—any metric suitable for classification problems can be used and this is decided by a modeler. In real projects AUC or F1-score are preferred as they are much more robust than classification error, but in this example for an easier understanding will include classification error).

By now, there has been estimated the error of the logistic regression model trained using outer training data of the first iteration with the optimal set of hyperparameters found in the inner loop and adjusted/retrained using CPLE and unlabeled data. This performance metric value (e.g. classification error) is the output of the first iteration of the outer loop. As an example this error could be 12%.

Iteration 2 of the Outer Cross-Validation

Now it is proceeded with the second iteration of the outer cross-validation: step (3) is started again, this time with new outer training and test set as illustrated in FIG. 14.

Steps (4-8) are performed just as described above, and the output of this second iteration of the outer cross-validation is another performance metric value, e.g. 8%.

Iteration 3 of the Outer Cross-Validation

Now it is proceeded with the third iteration of the outer cross-validation: step (3) is started again, this time with new outer training and test set as illustrated in FIG. 15.

For this new data division, again steps (4-8) are repeated and some estimate of the classification error is achieved, e.g. 15%.

In this illustration, an outer cross-validation with 5 iterations is presented (you already got the idea). In practice, one normally takes either 5 or 10 iterations. The more iterations (this also applies to the inner loop), the longer the execution time. A modeler must decide what is feasible number of iterations for both inner and outer cross-validation in each concrete project.

Iterations 4 and 5 are executed as well and measured the classification errors of e.g. 13% and 6%, respectively.

Step (9) averages these performance metric values over different folds:

| | |
|---|---|
| 1 | 12 |
| 2 | 8 |
| 3 | 15 |
| 4 | 13 |
| 5 | 6 |

Average error is then (12+8+15+13+6)/5=10.8%

Now the whole procedure described above is repeated for the next untrained model (from the list in step (3)). E.g. that model is Random forest. Then in the next iteration of the step (2), next model is evaluated, e.g. adaboost etc. until all the models are evaluated.

In step (10), the model with the smallest aggregated error is selected as the final model for the time horizon given in step (1):

| | |
|---|---|
| log. reg. | 10.8 |
| random forest | e.g. 7 |
| adaboost | e.g. 15 |
| . . . | . . . |

So the best model for the evaluated time horizon in this example is random forest as it has the smallest error (7%). Please recall that there were 5 iterations of the outer loop and each iteration produced one random forest model on the corresponding outer training set and evaluated that model on the outer test set. The question now is, which of these 5 models should be used in practice, e.g. deployed at the customer site.

The answer is step (11): a new final random forest model will be trained using all labeled data and all unlabeled data. This can be illustrated as shown in FIG. 16.

This corresponds to doing steps (4-7) using all the available data. There is no outer loop anymore—it was used just to tell us which model was the best for the given time horizon (in our example this was random forest). This will generate final random forest model for the given time horizon.

Then it is returned to step (1), the next time horizon relevant for the disease (e.g. 3 months) is selected and started with step (2) again to get the best final model for this new time horizon. Maybe this will be adaboost, maybe k-NN classifier—steps (2-10) will discover which one it is and then in step (11) a final model will be trained using all labeled and unlabeled data for the 3 months time horizon.

Then the same will be done for the next time horizon.

FIG. 16 displays the first iteration of the outer loop of the nested cross-validation which incorporates the CPLE framework for making use of the unlabeled EMR data EU. However, as described before, in the EMR-datasets the labels are missing for the large portion of instances. Nevertheless, these EMR-validation datasets could also be used for the nested cross-validation (see above explained step (7). This step (7) is based on the solution to problem (D) above.

As can be seen, the CPLE meta learner is embedded inside of the outer loop of the nested cross-validation. When evaluating an algorithm and its hyperparameters, for each fold of the outer nested cross-validation loop a grid search can be performed in the inner loop using labeled training data only to find the best supervised model. Then the CPLE meta learner is employed receiving the whole dataset: unlabeled EMR-data, labeled EMR-data EL training and the best so far obtained model (by a supervised classifier training and tuning). The CPLE labels the unlabeled instances and retrains the best model, which can then be evaluated on the labeled test set. This procedure is performed once for each of the iterations of the outer nested cross-validation loop. The results can then be aggregated by computing the mean and standard deviation of the selected performance metrics over all outer loop's iterations. Here, the first iteration of the outer loop in the proposed extension is illustrated in FIG. 16. The subsequent iterations are conceptually the same with the exception of the different outer and inner training and test/validation folds which changes in each iteration.

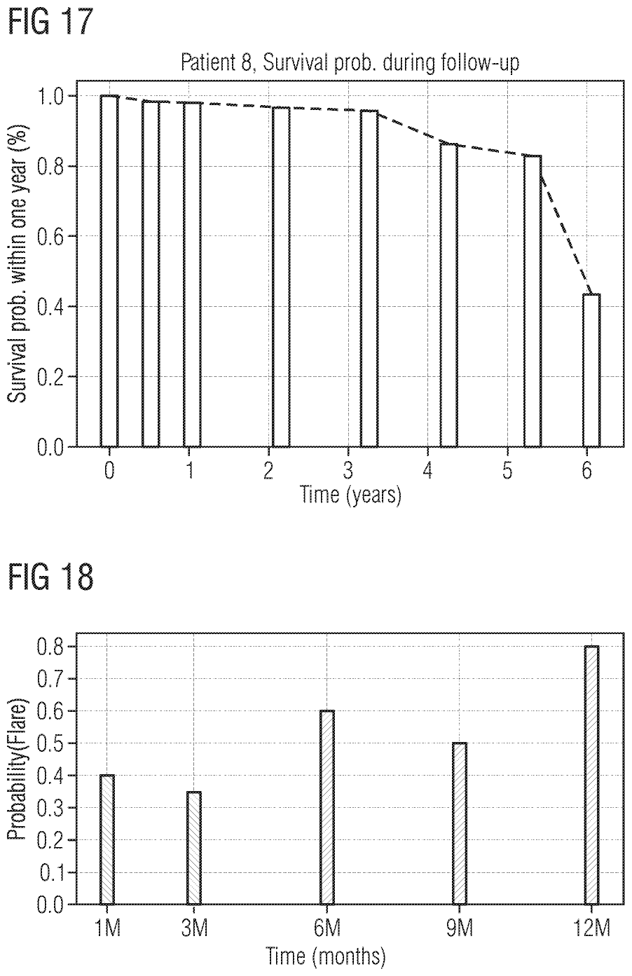

FIG. 17 displays visualized predicted survival probabilities (in general this can be any clinical event-free probability, not just death-free) within a time horizon of one year for a patient, given some medication and dose decided by a clinician. In this figure one can see that at the first seven follow-ups (first seven bars) the predicted survival probability within a year from the current follow-up was pretty high (about 80% to 100%). However, at the last follow-up at the 6th year of treatment (eighth bar), this probability dropped to under 50% and this patient actually died a couple of months after the last follow-up. If the clinician had such model and visualization of its predictions, he/she could have experimented with different treatments in order to try to prevent the adverse event.

FIG. 18 displays the predicted RA flare probabilities (in general this can be any clinical event probability) for an RA patient for five different time horizons computed at one patient visit for a given treatment. These predictions are computed by five different machine learning models which form a meta-model.

In this figure, the first two bars (shaded from left to right) illustrate predicted flare probability lower than 50% while the last three bars (shaded from right to left) mark flare probability higher or equal to 50% (this threshold can be decided by clinicians). Predictions are computed for five time horizons of interest (domain dependent; these time horizons might be relevant for the RA disease but can look differently for some other disease). So the first bar shows about 40% probability that the patient will experience a flare within 1 month from the current visit. The second bar shows about 35% probability that the patient will have a flare within 3 months from the current visit (with the same treatment as in the first time horizon of 1 month). The third bar shows probability over 50% that the patient will have a flare within 6 months from the current visit and so on. Such insight enables the clinician to evaluate how long the “flare-free” period would last for a given patient and a given treatment decision. Using this information, it can be estimated when the next patient visit would be necessary. For the example in FIG. 18, the clinician could order the next examination shortly before three months from the current visit expire because the predictive models for both 1 month and 3 months time horizons predict relatively low probability of the adverse event.

In addition to visualizing adverse event probabilities over past follow-ups of a single patient (FIG. 17) and computing and visualizing predictions for different time horizons (FIG. 18). The simulation strategy could be employed for various treatments (in RA case these are medications and dose) to compute adverse event probabilities for all prediction horizons of interest. This is illustrated in FIG. 19.

FIG. 19 displays recommendation system based on the multi-objective optimization. Inputs to the optimization problem are weighted event probabilities for various time horizons, follow-up necessity, as well as the monetary treatment expense. In this figure, the system rated clinician's options and may mark the three most promising ones with are the columns 4, 5 and the third from below in color.

The first three columns in FIG. 19 relate to the treatment (in this illustration the RA example is given). Here different medications and possible dosage options are listed. Since these represent an input to predictive models for different time horizons, probabilities of an adverse event (e.g. RA flare) are computed and graphically illustrated (column 4). Ideally, for all time horizons event probabilities should be minimized. Normalized expense of the treatment is shown in column 5. Normalization is done in a way that the most expensive drug and dosage correspond to the normalized expense of 1 and the zero dose corresponds to the normalized expense 0. Of course, one would want to minimize this criterion.

The column 6 relates to the normalized latest next follow-up. Depending on the predicted event probability, longest “safe” visit-free time is defined. The larger this time is, the better, i.e. it should be maximized if possible. The visit-free time is normalized to the interval [0,1] in the following way: if the event probability is high (e.g. >=50%, but this threshold can be different, depending on the application area of the proposed solution) already at the shortest time horizon of 1 month, then the longest “safe” normalized visit-free time is 0 for the given treatment. If however the predictions for all relevant time horizons are small (e.g. <50%), the longest visit-free period is 1 for the given treatment. Since this criterion is supposed to be maximized, we transform it into the minimization problem for consistency with other criteria by computing “1-normalized visit-free time”, which is given in column 6 in FIG. 19.

In the example above, there are five relevant predicted probabilities (in other examples or diseases there might be more or less of them), normalized monetary treatment expense and 1-normalized visit-free time as criteria that should be minimized in an optimization problem. As stated in point 1, different clinicians treat their patients in different ways. Some may be more liberal while the others may be more conservative. This means that different aforementioned criteria will have different weights/importance to different clinicians, i.e. some would weight risk of flaring within 3 months higher than the necessary follow-up frequency and for the others the budget also plays a very important role.

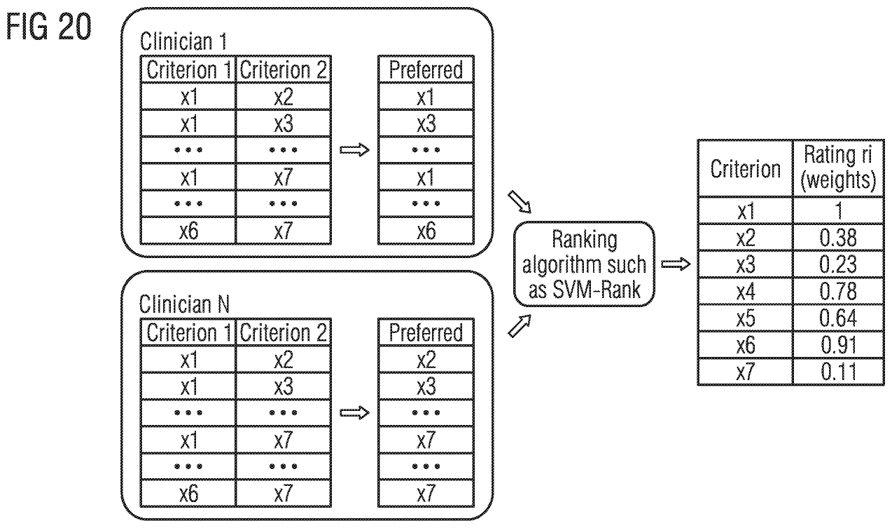

In order to offer common treatment recommendations which summarize approaches of different clinicians and take into account all 7 criteria, according to one of its embodiments the invention has the technical features: Each clinician in a department who treats patients (e.g. rheumatology department) performs pairwise comparisons of these criteria marking the one which is in their opinion more important. For 7 criteria, there are 7×6/2=21 pairwise comparison that need to be made. Assuming one needs about 10 seconds to decide which of the offered two criteria is more important to him/her, this procedure would take about 210 s=3.5 minutes per clinician. This is not a lot of time and should be feasible for every clinician to accomplish in a timely manner. In particular, a machine learning ranking algorithm such as the SVM-Rank (see e.g. “Training Linear SVMs in Linear Time”; T. Joachims, KDD '06, Aug. 20-23), the entire contents of which are hereby incorporated herein by reference, can be used which generates ratings from pairwise comparisons. This is illustrated in FIG. 20.

FIG. 20 displays the ranking procedure based on the SVM-Rank algorithm. N clinicians perform pairwise comparisons of the seven criteria (in other example/diseases there could be other, less or even more criteria). Clinicians' preferences are input to the algorithm which generates a rating of criteria. These ratings are used as weights in the optimization function to rank recommendations in the clinical decision support system.

Each of the seven criteria of above example would be assigned a rating (i.e. weight) based on the overall assessment of the criteria performed by clinicians. These weights are then normalized to the interval [0,1] where 1 is assigned to the most and 0 to the least important one.

Now the following technical feature can be defined as an optimization problem with the following inputs:

x1: probability of an event within one month
x2: probability of an event within three months
x3: probability of an event within six months
x4: probability of an event within nine months
x5: probability of an event within twelve months
x6: 1—normalized visit free time
x7: normalized expenses
and an optimization function to be minimized given as:

f(x₁,x₂, . . . ,x₇)=Σₖ₌₁⁷rᵢxᵢ (4)

Where rᵢ are weights obtained from the ranking algorithm and pairwise comparisons of the criteria.

For each treatment option that clinician can take, a treatment rating score (column 7 in FIG. 19) that is computed as a result of the function given above. Based on this score, a recommendation is derived (column 8) where three best recommendations are highlighted.

Finally, all aforementioned technical features and methods taken together represent a basis of the system for automated semi-supervised classification and treatment optimization of disease activity in autoimmune diseases using EMR data. This training phase of such system is illustrated in FIG. 21.

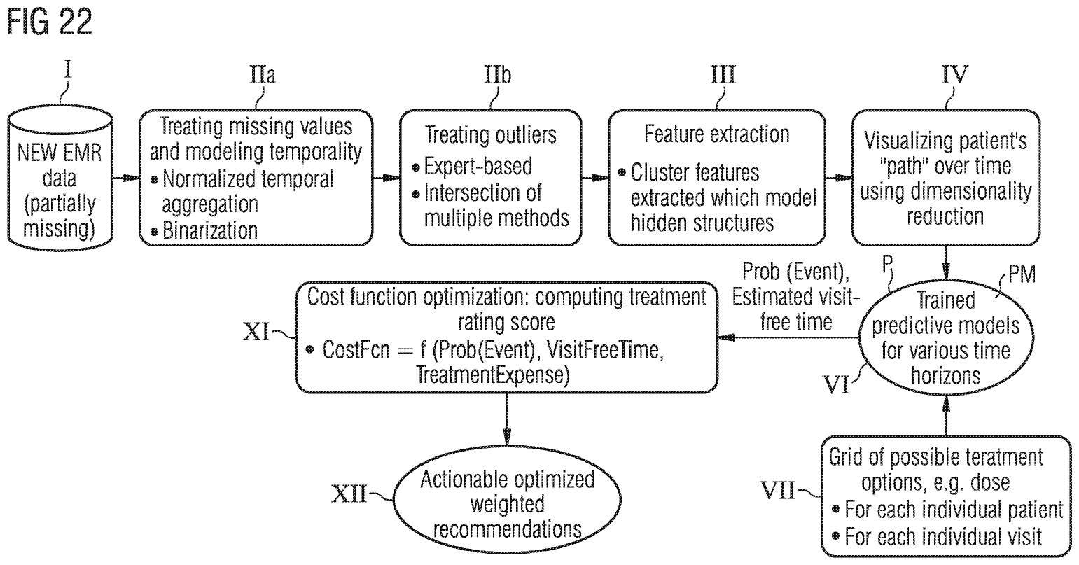

Once trained, the system would be deployed and applied in practice (productive phase). The components of the solution used in the productive phase are illustrated in FIG. 22.

FIG. 21 displays a system and/or a method for automated semi-supervised classification and treatment optimization of disease activity in autoimmune diseases using EMR datasets in the training phase.

In step I, a number of EMR-datasets comprising measurements and patient related data of a number of follow-ups are provided. In this example, some data are partially missing and there could be the case that EMR-data is not labeled (and thus not suitable for a nested cross validation concerning the state of the art). Thus, it is assumed that labeled EMR-data EL and unlabeled EMR-data EU is applied.

Step II, where there is performed a treating of the EMR-datasets in order to estimate missing values and/or to correct outliers and/or to model temporality of measurements, is here divided in two sub-steps IIa and IIb.

Step IIa comprises the action of Treating missing values and modeling temporality. This may include a normalized temporal aggregation and/or a binarization. The aggregation is used to reduce missing values. The normalization is used to preserve typical variable ranges.

Step IIb comprises the action of treating outliers. This could be e.g. a state-of-the-art outlier treatment that is typical analysis. This treatment should be expert-based and/or should comprise a intersection of multiple methods.

Now, the labeled EMR-data EL′ and the unlabeled EMR-data EU′ is adjusted and ready to be used by the nested cross-validation.

Step III comprises a target and feature extraction. Here two steps of the inventive method, i.e. forming subgroups of related follow ups within the EMR-datasets and providing a target-variable or extract a target-variable from the EMR-dataset, are performed. The target and feature extraction should include a binary target variable (or more) for different time horizons. The forming of subgroups should include the action of clustering features extracted with model hidden structures, e.g. in addition to static and dynamic patient data from the literature, cluster features extracted.

In step IV the patient's “path” over time is visualized using dimensionality reduction. This can be achieved in that the number of data dimensions in the EMR-datasets is reduced and the EMR-datasets in reduced number of dimensions are visualized. The number of data dimensions in the EMR-datasets is preferably reduced by using a Principal Component Analysis (“PCA”), wherein patient data of the EMR-datasets are especially represented in a PCA-space using two or three dimensions and wherein data from follow-ups that are associated with a clinical event are preferably represented in the PCA-space as points of one individual subgroup, wherein the subgroups are preferably visualized, especially in a space, where dangerous areas are marked.

Step V comprises predictive modeling. Here e.g. the probability of flares is estimated from the features (Prob(Flares)=g(Features)). This could be achieved with feature selection, hyperparameter optimization, nested X-validation (labeled data) and/or nested X-validation with embedded CPLE-framework (semi-supervised, non-labeled data), with the performance metrics: AUC, sensitivity, specificity, F1-score, etc.

This step comprises providing a number of untrained predictive models PM (e.g. logistic regression, linear discriminant analysis, quadratic discriminant analysis, decision tree, extra tree classifier, random forest, adaboost, gradient boost, bagging classifier, k-nearest neighbor, naïve Bayes classifier and support vector machine), which can output probabilities and assign weights to EMR-data, wherein each model is capable of being trained with data using methods of machine learning, providing a number of different time horizons, and performing a nested cross validation for each time horizon and for each predictive model PM. Here it is advantageous to apply an agnostic approach, an automated optimal model and parameter selection.

In step VI there is a number of predictive models PM for various time horizons that are readily trained.

To produce a result that can easily be understood by a user (and may comprise other valuable information), in step VII a grid of possible treatment options (e.g. dose) is added to the models. It is advantageous that this grid is created for each individual patient and/or for each individual visit.

In step VIII, a group of physicians (symbol in the circle) perform pairwise comparisons of criteria. The result of these comparisons is generated by a ranking machine algorithm such as SVM-Rank.

The ratings are given to a Routine of Additional Inputs. These additional inputs may be a value for treatment expenses (step X) or ratings (weights) of different criteria (Step IX) that are provided for a final generation of results.

In step XI the final result is generated by minimizing a cost function optimization: a computing treatment rating score. e.g. the probability of an event (Prob(event)), a value for the visit free time (VisitFreeTime), and expenses for a treatment (TreatmentExpense) could be part of this cost function (CostFcn):

CostFcn=f(Prob(event),VisitFreeTime,TreatmentExpense).

In step XII the result of this minimization are provided for a user in form of actionable optimized weighted recommendations.

FIG. 22 displays a system and/or a method for automated semi-supervised classification and treatment optimization of disease activity in autoimmune diseases using EMR data in the productive or application phase. Here Steps I to IV are performed like in above example, however not on old EMR-data, only but at least on an actual EMR-dataset (of a recent follow up) in order to treat the dataset the “right” way so that the trained predictive models are able to “understand” the data of the EMR-dataset.

In step VI there is a number of predictive models for various time horizons that are already trained (e.g. by a method as shown in FIG. 21).

To produce a result that can easily be understood by a user (and may comprise other valuable information), in step VII a grid of possible treatment options (e.g. dose) is added to the models. It is advantageous that this grid is created for each individual patient and/or for each individual visit.

In step XI the final result is generated by minimizing a cost function optimization: a computing treatment rating score. e.g. the probability of an event (Prob(event)), a value for the visit free time (VisitFreeTime), and expenses for a treatment (TreatmentExpense) could be part of this cost function (CostFcn):

CostFcn=f(Prob(event),VisitFreeTime,TreatmentExpense).

In step XII the result of this minimization are provided for a user in form of actionable optimized weighted recommendations.

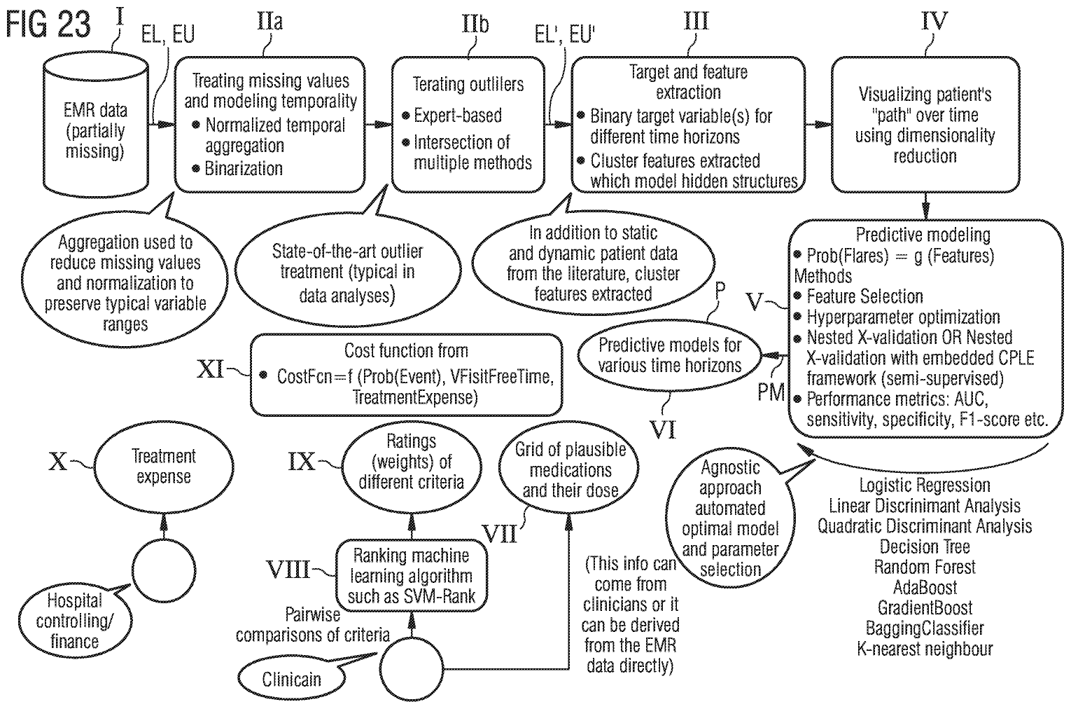

FIG. 23 displays a preferred method for creating predictive models for an automated clinical decision support system for automated supervised and semi-supervised classification and treatment optimization of clinical events using EMR data.

In step I, a number of EMR-datasets comprising measurements and patient related data of a number of follow-ups are provided. In this example, some data are partially missing and there could be the case that EMR-data is not labeled (and thus not suitable for a nested cross validation concerning the state of the art). Thus, it is assumed that labeled EMR-data EL and unlabeled EMR-data EU is applied.

Step II, where there is performed a treating of the EMR-datasets in order to estimate missing values and/or to correct outliers and/or to model temporality of measurements, is here divided in two sub-steps IIa and IIb.

Step IIa comprises the action of treating missing values and modeling temporality. This may include a normalized temporal aggregation and/or a binarization. The aggregation is used to reduce missing values. The normalization is used to preserve typical variable ranges.

Step IIb comprises the action of treating outliers. This could be e.g. a state-of-the-art outlier treatment that is typical analysis. This treatment should be expert-based and/or should comprise a intersection of multiple methods.

Now, the labeled EMR-data EL′ and the unlabeled EMR-data EU′ is adjusted and ready to be used by the nested cross validation.

Step III comprises a target and feature extraction. Here two steps of the inventive method, i.e. forming subgroups of related follow ups within the EMR-datasets and providing a target-variable or extract a target-variable from the EMR-dataset, are performed. The target and feature extraction should include a binary target variable (or more) for different time horizons. The forming of subgroups should include the action of clustering features extracted with model hidden structures, e.g. in addition to static and dynamic patient data from the literature, cluster features extracted.

In step IV the patient's “path” over time is visualized using dimensionality reduction. This can be achieved in that the number of data dimensions in the EMR-datasets is reduced and the EMR-datasets in reduced number of dimensions are visualized. The number of data dimensions in the EMR-datasets is preferably reduced by using a Principal Component Analysis (“PCA”), wherein patient data of the EMR-datasets are especially represented in a PCA-space using two or three dimensions and wherein data from follow-ups that are associated with a clinical event are preferably represented in the PCA-space as points of one individual subgroup, wherein the subgroups are preferably visualized, especially in a space, where dangerous areas are marked.

Step V comprises predictive modeling. Here e.g. the probability of flares is estimated from the features (Prob(Flares)=g(Features)). This could be achieved with feature selection, hyperparameter optimization, nested X-validation (labeled data) and/or nested X-validation with embedded CPLE-framework (semi-supervised, non-labeled data), with the performance metrics: AUC, sensitivity, specificity, F1-score, etc.

This step comprises providing a number of untrained predictive models (e.g. logistic regression, linear discriminant analysis, quadratic discriminant analysis, decision tree, extra tree classifier, random forest, adaboost, gradient boost, bagging classifier, k-nearest neighbor, naïve Bayes classifier and support vector machine), which can output probabilities and assign weights to EMR-data, wherein each model is capable of being trained with data using methods of machine learning, providing a number of different time horizons, and performing a nested cross validation for each time horizon and for each predictive model. Here it is advantageous to apply an agnostic approach, an automated optimal model and parameter selection.

In step VI there is a number of predictive models PM for various time horizons that are readily trained. If the circle of step VI would show a hardware unit, this would be a preferred prediction-unit P of at least one embodiment of the invention.

To produce a result that can easily be understood by a user (and may comprise other valuable information), in step VII a grid of plausible medications and their dose is added to the models. It is advantageous that this grid is created for each individual patient and/or for each individual visit. The information for the grid can come from clinicians (lowest round symbol) or it can be derived from the EMR-datasets directly.

In step VIII, a group of physicians (symbol in the circle) perform pairwise comparisons of criteria. The result of these comparisons is generated by a ranking machine algorithm such as SVM-Rank.

In Step IX, the ratings (weights) of different criteria are provided for a final generation of results.

In step X, values for treatment expenses are provided for a final generation of results e.g. by the board of hospital controlling/finance (left round symbol).

In step XI the final result is generated by minimizing a cost function optimization: a computing treatment rating score. e.g. the probability of an event (Prob(event)), a value for the visit free time (VisitFreeTime), and expenses for a treatment (TreatmentExpense) could be part of this cost function (CostFcn):

CostFcn=f(Prob(event),VisitFreeTime,TreatmentExpense).

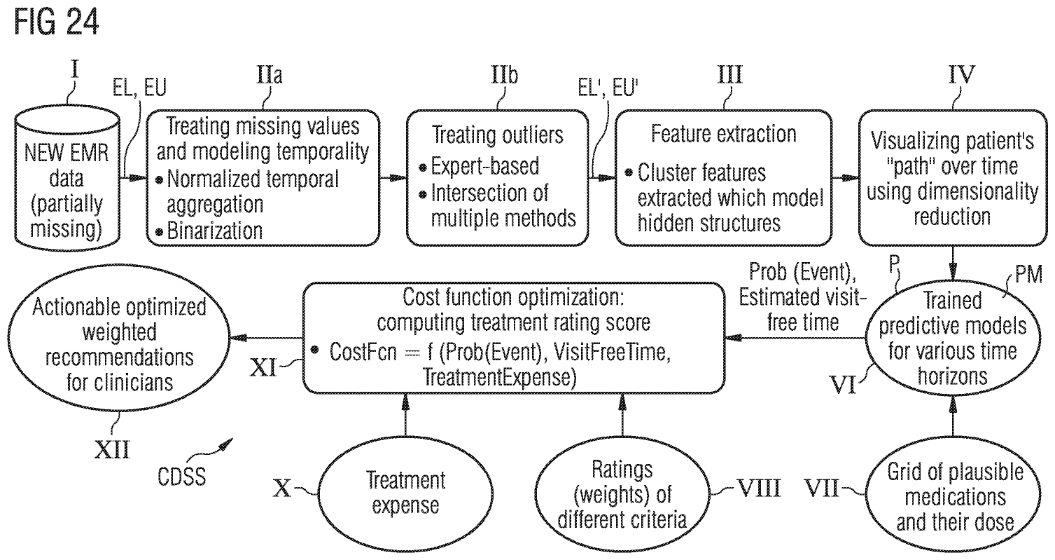

FIG. 24 displays a preferred method for automated clinical decision support for automated supervised and semi-supervised classification and treatment optimization of clinical events using EMR data. If the steps would be units performing these steps, this figure could also show a Clinical Decision Support System (CDSS).

Here Steps I to IV are performed like in above example, however not on old EMR-data, only but at least on an actual EMR-dataset (of a recent follow up) in order to treat the dataset the “right” way so that the trained predictive models are able to “understand” the data of the EMR-dataset.

In step VI there is a number of predictive models for various time horizons that are already trained (e.g. by a method as shown in FIG. 23).

In Step IX, the ratings (weights) of different criteria are provided for a final generation of results.

In step X, values for treatment expenses are provided for a final generation of results.

In step XI the final result is generated by minimizing a cost function optimization: a computing treatment rating score. e.g. the probability of an event (Prob(event)), a value for the visit free time (VisitFreeTime), and expenses for a treatment (TreatmentExpense) could be part of this cost function (CostFcn):

CostFcn=f(Prob(event),VisitFreeTime,TreatmentExpense).

In step XII the result of this minimization are provided for a user in form of actionable optimized weighted recommendations for clinicians.

In the diagrams, like numbers refer to like objects throughout. Objects in the diagrams are not necessarily drawn to scale.

FIG. 23 shows the training-phase and the collection of necessary information (what are the therapies cost, which drugs and which dose can be used theoretically and which criteria are more important to the doctors than the others). In this figure, there are four results that will be used when using the system, namely the meta-model (see step VI, comprising several prognostic models for different time periods), the ratings of the criteria in pairwise comparisons (see step IX), theoretically possible medication and dose for the disease (see step VII, comes from doctors or can be extracted directly from EMR data) as well as the cost of these drugs (see step X, come from the controlling department of a hospital).

These four results are used in the use of the system in practice (FIG. 24): The “Grid” of various possible therapies as well as the new EMR data are given to the meta-model, which then generates a forecast for the possibility of a clinical event (death, thrust, readmission etc.) for different periods (1, 3, 6, 9, 12 months). These possibilities are provided together with the estimated visit free time. The cost function also recognizes the ratings of the criteria and the costs of the respective therapies as inputs and calculates for each potential therapy from the grid a “treatment rating score” on the basis of which the therapies are evaluated and the best three therapies are suggested to the doctor.

Although the present invention has been disclosed in the form of preferred embodiments and variations thereon, it will be understood that numerous additional modifications and variations could be made thereto without departing from the scope of the invention.

For the sake of clarity, it is to be understood that the use of “a” or “an” throughout this application does not exclude a plurality, and “comprising” does not exclude other steps or elements. The mention of a “unit” or a “module” does not preclude the use of more than one unit or module.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for creating predictive models for an automated clinical decision support system for automated supervised and semi-supervised classification and treatment optimization of clinical events using EMR data, the method comprising:
providing a number of EMR-datasets including measurements and patient related data of a number of follow-ups;
providing a target-variable or extracting a target-variable from the EMR-datasets;
providing a number of untrained predictive models to output probabilities and assign weights to EMR-data of the EMR-datasets, wherein each predictive model, of the number of untrained predictive models, is trainable with data using methods of machine learning;
providing a number of different time horizons;
performing a nested cross-validation for each time horizon, of the number of different time horizons, and for each predictive model, of the number of untrained predictive models; and
selecting a respective predictive model for each respective time horizon based on the nested cross-validation performed.

2. The method of claim 1, wherein missing values in the EMR-datasets are estimated using a sliding weighting function, and wherein, thereafter, a binarization approach is applied to encode remaining missing values as binary zero vectors.

3. The method of claim 1,
wherein subgroups of related data from different follow-ups within the EMR datasets are formed by using a clustering algorithm, wherein the subgroups are exploited to generate predictors, by clustering follow-ups of patients in the EMR-datasets to subgroups,
wherein an optimal number of subgroups for a EMR-dataset, of the EMR datasets, is determined,
wherein the subgroups are used as predictors in the classification of clinical events such that, for each respective follow-up
a first feature is created from a cluster membership of each respective follow-up, and
other features are created from a Minkowski distance of each respective follow-up to a center of each subgroup.

4. The method of claim 1, wherein a number of data dimensions in the EMR-datasets is reduced and the EMR-datasets in reduced number of dimensions are visualized.

5. The method of claim 1, wherein the respective predictive models are selected from a group comprising logistic regression, linear discriminant analysis, quadratic discriminant analysis, decision tree, extra tree classifier, random forest, adaboost, gradient boost, bagging classifier, k-nearest neighbor, naïve Bayes classifier and support vector machine.

6. The method of claim 1,
wherein for each iteration of an outer loop of the nested cross-validation, a set of hyperparameters relevant for the predictive models is determined, wherein a grid of values is created for a number of most influential hyperparameters of each respective predictive model,
wherein for each set of hyperparameters, a predictive model is trained in an inner loop of the nested cross-validation using labeled EMR-data of the EMR-datasets of a current outer loop iteration, and wherein the method further comprises:
selecting an optimal hyperparameter set for a respective predictive model,
training an inner loop final model using all labeled EMR-data of the outer loop iteration and optimal hyperparameter set selected,
testing an optimal predictive model of the inner loop using labeled test data of the outer loop iteration,
aggregating test results of the outer loop iterations,
comparing aggregated testing results of the outer loop for different trained predictive models and selecting the optimal predictive model, and
training a final predictive model for a time horizon by repeating iterations of the outer loop for each respective predictive model using all labeled EMR-data.

7. The method of claim 1, wherein upon the EMR-datasets including unlabeled EMR-data, an inner loop optimal model of the nested cross-validation is retrained using a Contrastive Pessimistic Likelihood Estimation framework assigning soft-labels to unlabeled EMR-data.

8. A prediction unit for creating prediction-data for an automated clinical decision support system (CDSS) for automated supervised and semi-supervised classification and treatment optimization of clinical events using EMR datasets, the prediction unit comprising:
a number of trained prediction models, each of the number of trained prediction models being for a different time horizons, of a number of different time horizons for which a nested cross-validation is performed.

9. A method for automated clinical decision support for automated supervised and semi-supervised classification and treatment optimization of clinical events using EMR datasets, the method comprising:
providing a number of trained prediction models, trained by the method of claim 1;
providing an EMR-dataset of a patient including measurements and patient related data of a number of follow-ups including data of a present patient follow-up; and
calculating the probability of a clinical event in all relevant of the number of time horizons, with the number of trained prediction models.

10. The method of claim 9, comprising at least one of:
calculating and visualizing the probability of a clinical event for different at least one of medication and doses with the number of trained prediction models for each of the number of time horizons; and
estimating a time when no follow-up is necessary based on the calculating of the probability of a clinical event occurring.

11. The method of claim 9, further comprising:
computing ratings of included criteria using a ranking algorithm from pairwise comparisons of those criteria as provided by multiple clinicians.

12. A clinical Decision Support System for automated supervised and semi-supervised classification and treatment optimization of clinical events using EMR datasets, comprising:
the prediction unit of claim 8.

13. A method comprising:
using one or more methods for at least one of treating EMR-datasets and obtaining results from EMR-datasets, the one or more methods comprising:
a) estimating missing values in the EMR-datasets by using a sliding weighting function, wherein missing values are estimated from measurements of the EMR-datasets in a relevancy time window as a sum of weighted past known values;
b) modeling temporality of measurements in the EMR-datasets by value aggregation, wherein to fully account for temporality in the EMR-datasets, known measurements are replaced by the aggregated values;
c) forming subgroups of related data from different follow-ups within the EMR datasets using a clustering algorithm,
wherein an optimal number of subgroups for an EMR-dataset is determined,
wherein the subgroups are used as predictors in classification of clinical events such that, for each follow-up
a first feature is created from a cluster membership of the follow-up, and
other features are created from a Minkowski distance of the follow-up to a center of each subgroup;
d) reducing a number of data dimensions in the EMR-datasets and the EMR-datasets in reduced number of dimensions are visualized;
e) performing a nested cross-validation,
wherein for each iteration of an outer loop of the nested cross-validation, a set of hyperparameters relevant for predictive models,
wherein for each predefined set of hyperparameters, a predictive model is trained in an inner loop of the nested cross validation using labeled EMR-data of a current outer loop iteration,
followed by:
selecting an optimal hyperparameter set for a predictive model,
training the inner loop final model using all labeled EMR-data of the outer loop iteration and selected optimal hyperparameters,
testing the inner loop optimal predictive model using labeled test data of the outer loop iteration,
aggregating test results of outer loop iterations,
comparing aggregated testing results of the outer loop for different trained predictive models and selecting the optimal predictive model, and
training a final predictive model for a time horizon by repeating iterations of the outer loop for each predictive model using all labeled EMR-data and all unlabeled EMR-data;
f) upon the EMR-datasets including unlabeled EMR-data, retraining the inner loop optimal model of the nested cross validation using a Contrastive Pessimistic Likelihood Estimation framework assigning soft labels to unlabeled EMR-data, wherein for labeling, unlabeled EMR-data of the EMR-datasets apply:
creating a Supervised classifier by at least one of training and tuning based on labeled EMR-data from the EMR-dataset and a grid of hyperparameter values, by using the inner loop of a nested cross validation routine,
choosing soft-labels for unlabeled EMR-data randomly from a given interval of values,
creating a semi-supervised model by maximizing a CPL-function of the Contrastive Pessimistic Likelihood Estimation framework (CPLE) including a supervised model trained on the labeled data,
using the semi-supervised model for updating randomly chosen soft-labels, to maximize CPL-value,
repeating steps concerning the CPL-function and the CPL-value until convergence occurs;
g) calculating and visualizing probability of a clinical event in all relevant time horizons, with a number of trained prediction models for at least one of different medication and different doses with the number of trained prediction models for each time horizon;
h) estimating the time when no follow-up is necessary based on the calculation of the probability of a clinical event occurring,
i) obtaining financial costs for at least one of medication, doses, and a follow-up for a therapy; and
j) computing ratings of included criteria using a ranking algorithm from pairwise comparisons of those criteria as provided by multiple clinicians.

14. A non-transitory computer program product storing a computer program, directly loadable into a memory of a control unit of a computer system, the computer program including program elements for performing the method of claim 1 upon the computer program being executed by the control unit of the computer system.

15. A non-transitory computer-readable medium storing program elements, readable and executable by a computer unit to perform the method of claim 1 upon the program elements being executed by the computer unit.

16. The method of claim 1, further comprising:
treating the EMR-datasets to at least one of estimate missing values, correct outliers and model temporality of measurements.

17. The method of claim 1, further comprising:
forming subgroups of related follow ups within the EMR-datasets.

18. The method of claim 16, further comprising:
forming subgroups of related follow ups within the EMR-datasets.

19. The method of claim 2, wherein missing values in the EMR-datasets are estimated from measurements of the EMR-datasets in a relevancy time window as a sum of weighted past known values.

20. The method of claim 1, wherein temporality of measurements in the EMR-datasets are modeled explicitly by value aggregation, and wherein to fully account for temporality in the data, known measurements are replaced by the aggregated values.

21. The method of claim 4, wherein the number of data dimensions in the EMR-datasets is reduced by using a Principal Component Analysis (PCA).

22. The method of claim 21, wherein patient data of the EMR-datasets are represented in a PCA-space using two or three dimensions.

23. The method of claim 6, wherein
for each outer fold of the outer nested cross-validation loop, a grid search is performed in the inner loop using labeled training data to find a best supervised model, and
the best supervised model is retrained and then evaluated on labeled test set of the outer fold, wherein the method is repeated a number of times, once for each of a number of the iterations of the outer nested cross-validation loop.

24. The method of claim 7, wherein for labeling unlabeled EMR-data of the EMR-datasets, the method further includes:
creating a Supervised classifier by at least one of training and tuning based on labeled EMR-data from the EMR-dataset and a grid of hyperparameter values, using an inner loop of a nested cross-validation routine,
choosing soft-labels for unlabeled EMR-data randomly from an interval of values,
creating a semi-supervised model by maximizing a CPL-function of the Contrastive Pessimistic Likelihood Estimation framework (CPLE), including a supervised model trained on the labeled data,
using the semi-supervised model for updating randomly chosen soft-labels, to maximize a CPL-value, and
repeating steps concerning the CPL-function and the CPL-value until convergence occurs.

25. The method of claim 9, further comprising:
treating the EMR-dataset to at least one of estimate missing values, correct outliers and model temporality of measurements;
forming subgroups of related data given at different follow ups within the EMR-dataset and extracting cluster features; and
reducing a number of data dimensions in the EMR-dataset and visualizing data of the EMR-dataset in number of data dimensions reduced.

26. The method of claim 10, further comprising:
obtaining financial costs for at least one of medication, doses and a follow-up.

27. The method of claim 10, further comprising:
normalizing financial costs of various possible treatments;
computing 1-normalized visit-free time, wherein the normalized visit-free time is normalized to an interval based upon:
a) upon an event probability being high at a relatively shortest relevant time horizon, then a relatively longest “safe” normalized visit-free time is 0 for a treatment, and
b) upon the predictions for all relevant time horizons being relatively small, a relatively longest visit-free period is 1 for the treatment; and
computing a Treatment Rating Score for each plausible treatment as a sum of products of computed ratings for relevant criteria and quantitative values of the relevant criteria.

28. A non-transitory computer program product storing a computer program, directly loadable into a memory of a control unit of a computer system, the computer program including program elements for performing the method of claim 9 upon the computer program being executed by the control unit of the computer system.

29. A non-transitory computer-readable medium storing program elements, readable and executable by a computer unit to perform the method of claim 13 upon the program elements being executed by the computer unit.

30. A non-transitory computer program product storing a computer program, directly loadable into a memory of a control unit of a computer system, the computer program including program elements for performing the method of claim 9 upon the computer program being executed by the control unit of the computer system.

31. A non-transitory computer-readable medium storing program elements, readable and executable by a computer unit to perform the method of claim 13 upon the program elements being executed by the computer unit.
